(19) United States
(12) Patent Application Publication
(10) Pub. No.: US 2026/0015391 A1
(43) Pub. Date: Jan. 15, 2026

(54) COMPOSITIONS FOR HYPERVESICULATING GUT MICROBES AND THERAPEUTIC ENZYME DELIVERY AND METHODS OF USE THEREOF

(71) Applicants: Mario Feldman, St. Louis, MO (US); Evan Pardue, St. Louis, MO (US); Mariana Sartorio, St. Louis, MO (US); Biswanath Jana, St. Louis, MO (US)

(72) Inventors: Mario Feldman, St. Louis, MO (US); Evan Pardue, St. Louis, MO (US); Mariana Sartorio, St. Louis, MO (US); Biswanath Jana, St. Louis, MO (US)

(73) Assignee: Washington University, St. Louis, MO (US)

(21) Appl. No.: 19/265,401

(22) Filed: Jul. 10, 2025

Related U.S. Application Data
(60) Provisional application No. 63/669,542, filed on Jul. 10, 2024.

(51) Int. Cl. C07K 14/195; A61K 9/50; C12N 15/74

(52) U.S. Cl. CPC C07K 14/195; A61K 9/5068; C12N 15/74

(57) ABSTRACT

Engineered Bacteroides thetaiotaomicron (Bt) strains, including deletion mutant strains, and methods of use thereof are provided. Methods of delivering a therapeutic compound to a gut microbiome of a subject include generating an engineered Bacteroides thetaiotaomicron (Bt) strain to overproduce outer membrane vesicles (OMVs); loading a target therapeutic compound into the OMVs; and colonizing the gut microbiome of the subject with the engineered Bt strain. In some embodiments, the subject has at least one of lactose intolerance, phenylketonuria, inflammatory bowel disease, and a chronic intestinal condition.

### CROSS-REFERENCE TO RELATED APPLICATIONS

This application claims priority from U.S. Provisional Application Ser. No. 63/669,542 filed 10 Jul. 2024, which is incorporated herein by reference in its entirety.

### STATEMENT OF FEDERALLY SPONSORED RESEARCH OR DEVELOPMENT

This invention was made with government support under AI168719 awarded by the National Institutes of Health. The government has certain rights in the invention.

### MATERIAL INCORPORATED BY REFERENCE

The Sequence Listing, which is a part of the present disclosure, includes a computer readable form comprising nucleotide and/or amino acid sequences of the present invention (file name “020706-US-NP_2025-07-10_Sequence-Listing” created on 8 Jul. 2025; 64,991 bytes). The subject matter of the Sequence Listing is incorporated herein by reference in its entirety.

### FIELD

The present disclosure generally relates to delivery of therapeutics to the gut microbiome.

### BACKGROUND

Currently, there are very few remedies for chronic intestinal conditions, like lactose intolerance. Now, people can take Lactaid, and similar products, to provide an exogenous source of the enzyme lactase that is absent in people with lactose intolerance. However, the effects of this are short-lived, which prevents many from enjoying foods that contain dairy.

Vesiculation is a process by which cells utilize membranous compartments to traffic cellular contents. In eukaryotes, vesiculation, in the form of the trans-Golgi network, exosomes, and other extracellular vesicles, has been extensively studied. However, much less is known about vesiculation in bacteria. Outer Membrane Vesicles (OMVs) are small, spherical structures generated by the active blebbing of the outer membrane (OM) in gram-negative bacteria. Due to their OM origin, OMVs are composed of lipopolysaccharides (LPS), phospholipids, OM proteins, and periplasmic contents. OMVs have been studied in many gram-negative bacteria and are reported to mediate key bacterial processes, including pathogenesis, quorum sensing, immunomodulation, nutrient uptake, and envelope stress response. In spite of this, no general mechanisms for OMV biogenesis in gram-negative bacteria have been established. The current consensus is that vesicles can be derived from active processes or passive phenomena involving membrane destabilization. Recent reports have shown that explosive cell lysis is the primary source of vesicles in Pseudomonas aeruginosa. It is hinted that this process occurs in other closely related species, which suggests that OMVs may not be actively produced in these organisms. In contrast, a growing body of literature has demonstrated that OMV biogenesis in Bacteroides spp. is a highly regulated process. Nonetheless, the mechanism of OMV biogenesis in Bacteroidota remains poorly understood.

Bacteroides spp. are gut commensals that comprise ˜40% of the bacterial species in the human gastrointestinal tract. Bacteroides shape the intestinal environment by producing immunogenic compounds and degrading dietary- and host-derived glycans. Studies have shown that OMVs produced by Bacteroides spp. help facilitate many of their functions in the gut. Transmission electron microscopy (TEM) revealed that Bacteroides thetaiotaomicron (Bt) and Bacteroides fragilis (Bf) produce large quantities of OMVs of uniform size. Mass spectrometry (MS) analyses revealed that OMV protein cargo is primarily composed of lipoproteins that function as glycosidases or proteases, and this cargo is tailored according to the available glycan landscape. In addition, OMV-enriched lipoproteins contain a negatively charged motif (S-D/E3), called the Lipoprotein Export Sequence (LES), that is absent from OM-retained lipoproteins. These features are unprecedented and distinguish Bacteroides OMVs from the vesicles isolated by other gram-negative bacteria. By exploiting these characteristics, OM- and OMV-specific proteins were labeled with fluorescent markers to visualize OMV biogenesis in Bt. Fluorescence microscopy was employed to observe OMVs actively blebbing from the OM of Bt in the absence of cell lysis. Altogether, these findings demonstrate that OMV biogenesis in Bacteroides spp. is the result of an active, regulated process and not the result of cell lysis, as has been suggested for other bacteria.

### BRIEF DESCRIPTION OF THE DISCLOSURE

Among the various aspects of the present disclosure is the provision of therapeutic enzyme delivery via hypervesiculating gut microbes.

In accordance with an aspect of the present disclosure, an engineered Bacteroides thetaiotaomicron (Bt) strain is provided. The engineered Bt strain comprises at least one mutated dual membrane-spanning anti-sigma factor (Dma) protein.

In some embodiments, the at least one mutated Dma protein is selected from Dma1, Dma2, and Dma3; is selected from a BT_4721 mutation and a BT_1558 mutation; and/or comprises a deletion mutant. In some embodiments, the deletion mutant is Δdma1. In some embodiments, the engineered Bt strain further comprises a Dma-associated sigma factor (Das) protein. In some embodiments, the Das protein is selected from Das1, Das2, and Das3.

In accordance with another aspect of the present disclosure, a method for overproducing outer membrane vesicles (OMVs) is provided. The method comprising: modifying a Bacteroides thetaiotaomicron (Bt) strain to produce an engineered Bt strain, wherein the modification comprises at least one mutated dual membrane-spanning anti-sigma factor (Dma) protein.

In some embodiments, the at least one mutated Dma protein is selected from Dma1, Dma2, and Dma3; is selected from a BT_4721 mutation and a BT_1558 mutation; and/or comprises a deletion mutant. In some embodiments, the deletion mutant is Δdma1. In some embodiments of the method, the engineered Bt strain further comprises a Dma-associated sigma factor (Das) protein. In some embodiments, the Das protein is selected from Das1, Das2, and Das3.

In accordance with a further aspect of the present disclosure, a method of delivering a therapeutic compound to a gut microbiome of a subject is provided. The method comprising: culturing an engineered Bacteroides thetaiotaomicron (Bt) strain in a growth medium to overproduce outer membrane vesicles (OMVs); isolating the OMVs from the growth medium; loading a target therapeutic compound into the OMVs; and administering the loaded OMVs to the subject.

In some embodiments, the subject has at least one of lactose intolerance, phenylketonuria, inflammatory bowel disease, and a chronic intestinal condition. In some embodiments of the method, the engineered Bt strain comprises at least one of: a mutated dual membrane-spanning anti-sigma factor (Dma) protein selected from Dma1, Dma2, and Dma3; a Dma protein mutation selected from a BT_4721 mutation and a BT_1558 mutation; and a Dma protein deletion mutant. In some embodiments, the Dma protein deletion mutant is Δdma1. In some embodiments of the method, the engineered Bt strain further comprises a Dma-associated sigma factor (Das) protein. In some embodiments, the Das protein is selected from Das1, Das2, and Das3.

Other objects and features will be in part apparent and in part pointed out hereinafter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings described herein are for illustrative purposes only. The drawings are not intended to limit the scope of the present teachings in any way.

FIG. 1A is a Western blot against anti-polyHis showing TM and OMV fractions from BT WT constitutively expressing Inulinase fused to Nluc (INL-Nluc). This blot demonstrates that INL-Nluc is stably expressed and properly localizes to the OMV fraction. Samples were standardized by OD600 and ran on 10% SDS-PAGE.

FIG. 1B is a graph showing that filtered supernatants from cells expressing INL-Nluc display higher luminescent output than those expressing Nluc in the cytoplasm. Filtered supernatants from overnight cultures (˜20 hrs) were used for NanoGlow assays to quantify OMV production in vitro. Total luminescent output was normalized by OD600.

FIG. 1C is a schematic of OMV screening methodology.

FIG. 2A is a schematic showing that genomic sequencing of screening candidates revealed four independent transposon mutants in Dma1. Red dashes denote transposon insertions (1: 674nt, 2: 677nt, 3:1, 139nt, 4:1, 143nt).

FIG. 2B is a Western blot using anti-polyHis that shows that Dma1 transposon mutants contain more INL-Nluc in the OMV fraction than the wild-type. Samples were normalized by OD600 and run on 10% SDS-PAGE.

FIG. 2C is a set of transmission electron microscopy (TEM) images and graph that reveal that ΔDma1 produces significantly more OMVs than the wild-type. Left: Representative TEM images of OMVs from each strain. Right: Quantification of TEM images from each strain (FoV: Field of View). Three biological replicates of samples from each strain were fixed onto grids in triplicate (in materials and methods). Ten random images were taken from each grid (n=90 per strain) and OMVs were counted manually.

FIG. 2D is a Coomassie Blue Stain that demonstrates that BTΔ4721 produces significantly more OMVs than the WT.

FIG. 2E is an LPS Silver Stain that demonstrates that BTΔ4721 produces significantly more OMVs than the WT.

FIG. 2F is a Lowry Protein Assay that demonstrates that BTΔ4721 produces significantly more OMVs than the WT.

FIG. 2G is a pair of graphs demonstrating that ΔDma1 secretes significantly more membrane lipids as a result of hypervesiculation. Total lipids were extracted from TMs and OMVs from WT, ΔDma1, and ΔDma1Comp. In all cases, samples were normalized to OD600.

FIG. 3A is a Coomassie blue stain comparing protein profiles between WT, Δdas1, Δdma1, and Δdas1-dma1. This shows that deletion of Das1 in the Δdma1 background re-stores WT levels of vesiculation. Samples were normalized by OD600 values and run on 10% SDS-PAGE gel.

FIG. 3B is a schematic of constructs used for targeted pulldown assay.

FIG. 3C is a pair of Western blots using anti-polyHis (Green) and anti-GST (Red). Top: Gel shows that equivalent amounts of bait and prey proteins were mixed prior to passage through a column containing anti-GST resin. Bottom: Gel showing the proteins present after the elution step, which demonstrates that the N terminus of Dma1 is sufficient to sequester Das1.

FIG. 4A is a Volcano plot representation of transcriptome and data comparing BT WT and BTΔDma1.

FIG. 4B is a Volcano plot representation of proteome data comparing BT WT and BTΔDma1.

FIG. 4C is a Coomassie Blue stain of TM and OMV fractions isolated from 1. WT, 2. ΔDma1, 3. Δ1287/Dma1, 4. ΔNigD1/Dma1, 5. ΔNigD2/Dma1. Left: Protein profiles of mutant TMs are the same as WT, indicating that all changes are specific to the OMV fraction. Right: Deletion of NigD1 in the BTΔDma1 background restores the protein profile to WT. Samples were normalized by OD600 and run on 10% SDS-PAGE.

FIG. 5A is a schematic of the AlphaFold predicted structure of Dma1.

FIG. 5B is a schematic of the proposed orientation of Dma1 in the membrane of Bt (created with BioRender).

FIG. 5C is a Western blot against anti-polyHis from WCs constitutively expressing 1. Dma1 and Das1 together, 2. Das1 alone, and 3. empty vector control. Bands adjacent to stars (*) are non-specific bands. Demonstrates that full-length Dma1 is present in WCs, and the state of the protein is growth-phase dependent.

FIG. 5D is a set of Western blots of WCs, TMs, and OMVs collected from Bt strain constitutively expressing Dma1 containing a C-terminal His tag and an N-terminal Flag tag. Green channel is anti-polyHis, while the red channel is anti-Flag. Full-length Dma1 is present solely in the WC and TM fraction.

FIG. 6A is a schematic of Dma1, Dma2, and Dma3 protein structures. Genome analysis identified two additional proteins, Dma2 and Dma3, with structural similarity to Dma1. AlphaFold predicted structure of each Dma, along with their respective operons, are presented.

FIG. 6B is a maximum likelihood phylogenetic tree that was constructed from twenty-nine core genomes of various Bacteroidota. Amino acid sequences of Dma1 (WP_008766767.1), Dma2 (WP_008762208.1), Dma3 (WP_011108473.1) from Bt served as references for identification in the other genomes.

FIG. 7 is a schematic showing that in the described model, Dma1 and Dma2 sense extracellular stimuli and/or perturbations in the OM. This leads to the proteolytic degradation of the proteins to release their cognate sigma factors, and subsequently modulate gene expression to induce OMV biogenesis. Dma1/Das1-mediated hypervesiculation is dependent upon NigD1, while the regulon of Dma2 is unclear.

FIG. 8 is a graph showing that hyper- and hypovesiculating strains were identified during OMV screening. Nano-Glo assays were performed using filtered supernatants isolated from each potential candidate. Total luminescent output of each candidate was standardized by OD600, then normalized to the wild-type. Candidates exhibiting a 1.5-fold increase in luminescence were considered hypervesiculating, while those displaying a 0.5-fold decrease were deemed hypovesiculating.

FIG. 9A is a Western blot using whole cells (WCs) to check the expression of the OMV reporter.

FIG. 9B is a Western blot using isolated TMs and OMVs to check partitioning of the OMV reporter.

FIG. 9C is a group of protein profiles of OMVs from each transposon mutant were analyzed by SDS-PAGE followed by Coomassie staining. 1: WT, 2: BD.D9, 3: BD.E5, 4: BE.C11, 5: BF.A12, 6: BF.G10, 7: BF.D12.

FIG. 9D is an image wherein relative abundance of LPS content of transposon mutants were analyzed by SDS-PAGE followed by LPS silver staining. 1: E. coli 0111: B4, 2: WT, 3: BD.E5, 4: BE.C11, 5: BF.A12, 6: BF.G10, 7: BF.D12.

FIG. 10 is a graph showing that mutation of dma1 does not significantly impact growth in vitro. Growth curves performed in BHI media for WT, Δdma1, and its corresponding complemented strain.

FIG. 11A is a set of graphs wherein total lipids were isolated from TM fractions from WT, Δdma1, and Δdma1Comp. Lipids were relativized to an 18:0-20:4 phosphoinositol internal standard (IS). Red squares denote sphingolipids, dihydroceramides (DHC), ethanolamine-phosphoceramides (EPC), inositolphosphoceramide (IPC). Blue squares are phospholipids, phosphatidylethanolamine (PE), phosphatidylserine (PS), and phosphatidylinositol (PI). Yellow squares represent amino lipids, glycylserine dipeptide lipids (GS), glycylserine phosphoryl diacylglycerol (GS-PA), N-(3-O-Acyl) acyl glycylserine phosphoryl dihydroceramide (GS-PDHC).

FIG. 11B is a set of graphs wherein total lipids were isolated from OMV fractions from WT, Δdma1, and Δdma1Comp. Lipids were relativized to an 18:0-20:4 phosphoinositol internal standard (IS). Red squares denote sphingolipids, dihydroceramides (DHC), ethanolamine-phosphoceramides (EPC), inositolphosphoceramide (IPC). Blue squares are phospholipids, phosphatidylethanolamine (PE), phosphatidylserine (PS), and phosphatidylinositol (PI). Yellow squares represent amino lipids, glycylserine dipeptide lipids (GS), glycylserine phosphoryl diacylglycerol (GS-PA), N-(3-O-Acyl) acyl glycylserine phosphoryl dihydroceramide (GS-PDHC).

FIG. 12 is a graph showing that protein composition of subcellular fractions is consistent between the WT and Δdma1. Principal component analysis (PCA) of WC (stars), TM (squares), and OMV (circles) proteomes from Bt grown in BHI media. Four biological replicates were performed for each condition.

FIG. 13A is a Volcano plot representation of proteins enriched in the TM and OMV fractions in WT. Integral membrane proteins are represented in blue, lipoproteins with LES motifs are indicated in red, lipoproteins lacking the LES motif are depicted in yellow, and soluble proteins are indicated in dark gray.

FIG. 13B is a Volcano plot representation of proteins enriched in the TM and OMV fractions in Δdma1. Integral membrane proteins are represented in blue, lipoproteins with LES motifs are indicated in red, lipoproteins lacking the LES motif are depicted in yellow, and soluble proteins are indicated in dark gray. (A) OMV cargo selection is maintained in Δdma1, indicating that these OMVs are not the result of cell lysis.

FIG. 14 is a pair of Western blots showing that partitioning of OMV cargo is maintained in Δdma1. Western blots using anti-polyHis against different membrane and OMV enriched proteins (Top: BT_0587, Bottom: SusG (BT_3698)). These demonstrate that partitioning of proteins between the OM and OMVs is not aberrant in Δdma1.

FIG. 15 is a schematic of the amino acid sequence alignment of Dma1 and Bf Reo with consensus (Dma1, SEQ ID NO: 68; Reo, SEQ ID NO: 69; Consensus, SEQ ID NO: 70). Dma1 show 56% sequence identity with Reo. Amino acids highlighted in red represent high consensus levels (90%), while those in blue represent low consensus (50%). The predicted anti-sigma domain is shown in brackets. Alignment was done using MultAlin version 5.4.1.

FIG. 16 is a graph showing that mutation of das1 does not impact growth in the wild-type or Δdma1 background in vitro. Growth curves performed in BHI media for WT, Δdas1, Δdas1-dma1 and their corresponding complemented strain.

FIG. 17 is a set of images showing that mutants in Δdas1 and Δdma1 are not attenuated in aerobic stress. Aerobic exposure stress tests comparing WT, Δdas1, Δdma1, and their corresponding complemented strains incubated in air for different times. Strains were cultured overnight, then diluted to the equivalent of OD600-0.1 for the initial spot. Each subsequent spot is a ten-fold dilution of the previous one.

FIG. 18A is a schematic of gene synteny of Bt Dma1. Gene synteny for different Bacteroidota were compared to Dma1 (BT_4721) from Bt. Genes of the same color are conserved in different species, while those in grey differ.

FIG. 18B is a schematic of gene synteny of Bt Dma2. Gene synteny for different Bacteroidota were compared to Dma2 (BT_1558) from Bt. Genes of the same color are conserved in different species, while those in grey differ.

FIG. 18C is a schematic of gene synteny of Bt Dma3. Gene synteny for different Bacteroidota were compared to Dma3 (BT_2778) from Bt. Genes of the same color are conserved in different species, while those in grey differ.

FIG. 19 is a Coomassie Blue stain showing that Δdma2 induces OMV biogenesis in a similar manner to Δdma1. Coomassie Blue stain comparing protein profiles between WT, Δdma2, Δdas2, Δdma2-das2, and Δdma3. This gel shows that mutation of dma2 induces vesiculation, and this phenotype is dependent on its cognate sigma factor, Das2. Samples were normalized by OD600 values and run on 10% SDS-PAGE gel.

### DETAILED DESCRIPTION OF THE DISCLOSURE

The present disclosure is based, at least in part, on the discovery that colonizing the gut microbiome with Bacteroides spp. that produce OMVs containing the described engineered lactose fusion protein, provides longer-term relief from chronic intestinal conditions (such as lactose intolerance) compared to current exogenously-sourced treatments since these bacteria are common residents of the human gut microbiota. This technology further enables treatment of additional conditions, like phenylketonuria and inflammatory bowel diseases. It was previously discovered that the lipoprotein export sequence (LES) in Bacteroides thetaiotaomicron (Bt) can be utilized as a means to target foreign proteins into outer membrane vesicles (OMVs) that shape the intestinal environment. As a gut commensal, Bt is an ideal probiotic microbe to deliver therapeutic enzymes in the gut because they produce large quantities of OMVs engineered herein to have specific cargo. In exemplary embodiments, it was identified that mutation of BT_1558 and BT_4721 leads Bt to hypervesiculate. These hypervesiculating strains can be engineered with therapeutic enzymes targeted to their OMVs to treat chronic gut conditions, like lactose intolerance, phenylketonuria, and inflammatory bowel diseases. These types of hypervesiculating strains are advantageous for treating intestinal conditions because they can effectively increase and maintain the concentration of bioengineered therapeutics in the gut.

### Modulation Agents

As described herein, gene and/or associated protein expression has been implicated in various diseases, disorders, and conditions. As such, modulation of gene and protein expression can be used for treatment of such conditions. A modulation agent can modulate response, such as by inducing or inhibiting gene and/or protein expression signaling. Modulation can comprise modulating protein expression on cells, modulating the quantity of gene/protein expressing cells, or modulating the quality of gene/protein expressing cells.

Modulation agents can be any composition or method that modulates expression on cells. For example, a modulation agent can be an activator, an inhibitor, an agonist, or an antagonist. As another example, the modulation can be the result of gene editing.

A modulation agent can be an antibody (e.g., a monoclonal antibody). A modulating agent can be an agent that induces or inhibits progenitor cell differentiation into gene/protein expressing cells.

Signal Reduction, Elimination, or Inhibition by Small Molecule Inhibitors, shRNA, siRNA, or ASOs

As described herein, a modulation agent can be used for use in various therapies, such as to reduce/eliminate or enhance/increase expression signals. For example, a modulation agent can be a small molecule inhibitor, a short hairpin RNA (shRNA), or a short interfering RNA (siRNA). As another example, RNA (e.g., long noncoding RNA (lncRNA)) can be targeted with antisense oligonucleotides (ASOs) as a therapeutic. Processes for making ASOs targeted to RNAs are well known; see e.g., Zhou et al. 2016 Methods Mol Biol. 1402:199-213. Except as otherwise noted herein, therefore, the process of the present disclosure can be carried out in accordance with such processes.

### Inhibiting Agent

Inhibition of agents as described herein can be determined by standard pharmaceutical procedures in assays or cell cultures for determining the IC₅₀. The half maximal inhibitory concentration (IC₅₀) is a measure of the potency of a substance in inhibiting a specific biological or biochemical function. The IC₅₀ is a quantitative measure that indicates how much of a particular inhibitory substance (e.g., pharmaceutical agent or drug) is needed to inhibit, in vitro, a given biological process or biological component by 50%. The biological component could be an enzyme, cell, cell receptor, or microorganism, for example. IC₅₀ values are typically expressed as molar concentration. IC₅₀ is generally used as a measure of antagonist drug potency in pharmacological research. IC₅₀ is comparable to other measures of potency, such as EC₅₀ for excitatory drugs. EC₅₀ represents the dose or plasma concentration required for obtaining 50% of a maximum effect in vivo. IC₅₀ can be determined with functional assays or with competition binding assays.

### Chemical Agent

Examples of therapeutic agents are described herein and can include one or more therapeutic compounds/proteins/enzymes (e.g., a lactose fusion protein), or pharmaceutically acceptable salts thereof, deliverable via OMV (such as in a Bacteroides thetaiotaomicron (Bt) strain).

The formulas, analogs, and R groups can be optionally substituted or functionalized with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; a C₂-10cycloalkyl optionally containing unsaturation or one oxygen or nitrogen atom; straight chain or branched C₁₋₁₀alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein the unsubstituted phenyl ring or substituted phenyl ring can be optionally substituted with one or more groups independently selected from the group consisting of hydroxyl; C₁₋₁₀alkyl hydroxyl; amine; C₁₋₁₀carboxyl; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂-10cycloalkyl optionally containing unsaturation or one oxygen or nitrogen atom; straight chain or branched C₁₋₁₀alkyl amine; heterocyclyl; heterocyclic amine; aryl comprising a phenyl; and heteroaryl containing from 1 to 4 N, O, or S atoms; and the unsubstituted heterocyclyl or substituted heterocyclyl can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂-10cycloalkyl optionally containing unsaturation or one oxygen or nitrogen atom; heterocyclyl; straight chain or branched C₁₋₁₀alkyl amine; heterocyclic amine; and aryl comprising a phenyl; and heteroaryl containing from 1 to 4 N, O, or S atoms. Any of the above can be further optionally substituted.

The term “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include-OH. The “hydroxyl” can be optionally substituted.

The terms “halogen” and “halo”, as used herein, unless otherwise indicated, include a chlorine, chloro, Cl; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, include a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C₁₋₁₀ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or −3-methyl-1 butynyl. An alkyl can be saturated, partially saturated, or unsaturated. The “alkyl” can be optionally substituted.

The term “carboxyl”, as used herein, unless otherwise indicated, can include a functional group consisting of a carbon atom double bonded to an oxygen atom and single bonded to a hydroxyl group (—COOH). The “carboxyl” can be optionally substituted.

The term “carbonyl”, as used herein, unless otherwise indicated, can include a functional group consisting of a carbon atom double-bonded to an oxygen atom (C═O). The “carbonyl” can be optionally substituted.

The term “alkenyl”, as used herein, unless otherwise indicated, can include alkyl moieties having at least one carbon-carbon double bond wherein alkyl is as defined above and including E and Z isomers of said alkenyl moiety. An alkenyl can be partially saturated or unsaturated. The “alkenyl” can be optionally substituted.

The term “alkynyl”, as used herein, unless otherwise indicated, can include alkyl moieties having at least one carbon-carbon triple bond wherein alkyl is as defined above. An alkynyl can be partially saturated or unsaturated. The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O-isobutylenyl, —O-1-pentenyl, —O-2-pentenyl, —O-3-methyl-1-butenyl, —O-2-methyl-2-butenyl, -O-2,3-dimethyl-2-butenyl, —O-1-hexyl, —O-2-hexyl, —O-3-hexyl, —O-acetylenyl, —O-propynyl, —O-1-butynyl, —O-2-butynyl, —O-1-pentynyl, —O-2-pentynyl and —O-3-methyl-1-butynyl, —O-cyclopropyl, —O-cyclobutyl, —O-cyclopentyl, —O-cyclohexyl, —O-cycloheptyl, —O-cyclooctyl, —O-cyclononyl and —O-cyclodecyl, —O—CH₂-cyclopropyl, —O—CH₂-cyclobutyl, —O—CH₂-cyclopentyl, —O—CH₂-cyclohexyl, —O—CH₂-cycloheptyl, —O—CH₂-cyclooctyl, —O—CH₂-cyclononyl, —O—CH₂-cyclodecyl, —O—(CH₂)₂-cyclopropyl, —O—(CH₂)₂-cyclobutyl, —O—(CH₂)₂-cyclopentyl, —O—(CH₂)₂-cyclohexyl, —O—(CH₂)₂-cycloheptyl, —O—(CH₂)₂-cyclooctyl, —O—(CH₂)₂-cyclononyl, or —O—(CH₂)₂-cyclodecyl. An alkoxyl can be saturated, partially saturated, or unsaturated. The “alkoxyl” can be optionally substituted.

The term “cycloalkyl”, as used herein, unless otherwise indicated, can include an aromatic, a non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C₃-10 cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and cyclooctadienyl. The term “cycloalkyl” also can include-lower alkyl-cycloalkyl, wherein lower alkyl and cycloalkyl are as defined herein. Examples of -lower alkyl-cycloalkyl groups include, but are not limited to, —CH₂-cyclopropyl, —CH₂-cyclobutyl, —CH₂-cyclopentyl, —CH₂-cyclopentadienyl, —CH₂-cyclohexyl, —CH₂-cycloheptyl, or —CH₂-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S, and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “heterocyclic” can be optionally substituted.

The term “indole”, as used herein, is an aromatic heterocyclic organic compound with formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, can include a-CN group. The “cyano” can be optionally substituted.

The term “alcohol”, as used herein, unless otherwise indicated, can include a compound in which the hydroxyl functional group (—OH) is bound to a carbon atom. In particular, this carbon center should be saturated, having single bonds to three other atoms. The “alcohol” can be optionally substituted.

The term “solvate” is intended to mean a solvate form of a specified compound that retains the effectiveness of such compound. Examples of solvates include compounds of the invention in combination with, for example, water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “UM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatograph. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion, or another counterion. The counterion may be any organic or inorganic moiety that stabilizes the charge on the parent compound. Furthermore, a pharmaceutically acceptable salt may have more than one charged atom in its structure. In instances where multiple charged atoms are part of the pharmaceutically acceptable salt, the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further can include a stoichiometric or non-stoichiometric amount of water bound by non-covalent intermolecular forces.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The term “transfection,” as used herein, refers to the process of introducing nucleic acids into cells by non-viral methods. The term “transduction,” as used herein, refers to the process whereby foreign DNA is introduced into another cell via a viral vector.

The terms “heterologous DNA sequence”, “exogenous DNA segment”, or “heterologous nucleic acid”, “transgene”, “exogenous polynucleotide” as used herein, each refers to a sequence that originates from a source foreign (e.g., non-native) to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Sequences described herein can also be the reverse, the complement, or the reverse complement of the nucleotide sequences described herein. The RNA goes in the reverse direction compared to the DNA, but its base pairs still match (e.g., G to C). The reverse complementary RNA for a positive strand DNA sequence will be identical to the corresponding negative strand DNA sequence. Reverse complement converts a DNA sequence into its reverse, complement, or reverse-complement counterpart.

| Base | Name | Bases Represented | Complementary Base |
|---|---|---|---|
| A | Adenine | A | T |
| T | Thymidine | T | A |
| U | Uridine(RNA only) | U | A |
| G | Guanidine | G | C |
| C | Cytidine | C | G |
| Y | pYrimidine | C T | R |
| R | puRine | A G | Y |
| S | Strong(3Hbonds) | G C | S* |
| W | Weak(2Hbonds) | A T | W* |
| K | Keto | T/U G | M |
| M | aMino | A C | K |
| B | not A | C G T | V |
| D | not C | A G T | H |
| H | not G | A C T | D |
| V | not T/U | A C G | B |
| N | Unknown | A C G T | N |

Complementarity is a property shared between two nucleic acid sequences (e.g., RNA, DNA), such that when they are aligned antiparallel to each other, the nucleotide bases at each position will be complementary. Two bases are complementary if they form Watson-Crick base pairs.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

An “expression vector”, otherwise known as an “expression construct”, is generally a plasmid or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as enhancer and/or promoter regions and lead to efficient transcription of the gene carried on the expression vector. The goal of a well-designed expression vector is the efficient production of protein, and this may be achieved by the production of significant amount of stable messenger RNA, which can then be translated into protein. The expression of a protein may be tightly controlled, and the protein is only produced in significant quantity when necessary through the use of an inducer, in some systems however the protein may be expressed constitutively. As described herein, Escherichia coli is used as the host for protein production, but other cell types may also be used.

In molecular biology, an “inducer” is a molecule that regulates gene expression. An inducer can function in two ways, such as:
(i) By disabling repressors. The gene is expressed because an inducer binds to the repressor. The binding of the inducer to the repressor prevents the repressor from binding to the operator. RNA polymerase can then begin to transcribe operon genes. An operon is a cluster of genes that are transcribed together to give a single messenger RNA (mRNA) molecule, which therefore encodes multiple proteins.
(ii) By binding to activators. Activators generally bind poorly to activator DNA sequences unless an inducer is present. An activator binds to an inducer and the complex binds to the activation sequence and activates target gene. Removing the inducer stops transcription. Because a small inducer molecule is required, the increased expression of the target gene is called induction.

Repressor proteins bind to the DNA strand and prevent RNA polymerase from being able to attach to the DNA and synthesize mRNA. Inducers bind to repressors, causing them to change shape and preventing them from binding to DNA. Therefore, they allow transcription, and thus gene expression, to take place.

For a gene to be expressed, its DNA sequence (or polynucleotide sequence) must be copied (in a process known as transcription) to make a smaller, mobile molecule called messenger RNA (mRNA), which carries the instructions for making a protein to the site where the protein is manufactured (in a process known as translation). Many different types of proteins can affect the level of gene expression by promoting or preventing transcription. In prokaryotes (such as bacteria), these proteins often act on a portion of DNA known as the operator at the beginning of the gene. The promoter is where RNA polymerase, the enzyme that copies the genetic sequence and synthesizes the mRNA, attaches to the DNA strand.

Some genes are modulated by activators, which have the opposite effect on gene expression as repressors. Inducers can also bind to activator proteins, allowing them to bind to the operator DNA where they promote RNA transcription. Ligands that bind to deactivate activator proteins are not, in the technical sense, classified as inducers, since they have the effect of preventing transcription.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “ribosome binding site”, or “ribosomal binding site (RBS)”, refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs.

A ribosomal skipping sequence (e.g., 2A sequence such as furin-GSG-T2A) can be used in a construct to prevent covalently linking translated amino acid sequences.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using self-replicating primers, paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5 (9), 680-688; Sanger et al. (1991) Gene 97 (1), 119-123; Ghadessy et al. (2001) Proc Natl Acad Sci USA 98 (8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

“Point mutation” refers to when a single base pair is altered. A point mutation or substitution is a genetic mutation where a single nucleotide base is changed, inserted, or deleted from a DNA or RNA sequence of an organism's genome. Point mutations have a variety of effects on the downstream protein product-consequences that are moderately predictable based upon the specifics of the mutation. These consequences can range from no effect (e.g., synonymous mutations) to deleterious effects (e.g., frameshift mutations), with regard to protein production, composition, and function. Point mutations can have one of three effects. First, the base substitution can be a silent mutation where the altered codon corresponds to the same amino acid. Second, the base substitution can be a missense mutation where the altered codon corresponds to a different amino acid. Or third, the base substitution can be a nonsense mutation where the altered codon corresponds to a stop signal. Silent mutations result in a new codon (a triplet nucleotide sequence in RNA) that codes for the same amino acid as the wild type codon in that position. In some silent mutations the codon codes for a different amino acid that happens to have the same properties as the amino acid produced by the wild type codon. Missense mutations involve substitutions that result in functionally different amino acids; these can lead to alteration or loss of protein function. Nonsense mutations, which are a severe type of base substitution, result in a stop codon in a position where there was not one before, which causes the premature termination of protein synthesis and can result in a complete loss of function in the finished protein.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by 11e, Leu by 11e, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tm) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA: DNA sequence can be determined using the following formula: Tₘ=81.5° C.+16.6 (log₁₀ [Na⁺])+0.41 (fraction G/C content)−0.63 (% formamide)−(600/l). Furthermore, the Tₘ of a DNA: DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged | K R H |
| (Basic): | |
| Negatively Charged | D E |
| (Acidic): | |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), single guide RNA (sgRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14 (12): 807-15, describing targeting deoxyribonucleotide sequences; Lee et al. (2006) Curr Opin Chem Biol. 10, 1-8, describing aptamers; Reynolds et al. (2004) Nature Biotechnology 22 (3), 326-330, describing RNAi; Pushparaj and Melendez (2006) Clinical and Experimental Pharmacology and Physiology 33 (5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-IT TM RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Genome Editing

As described herein, gene and/or protein expression signals can be modulated (e.g., reduced, eliminated, or enhanced) using genome editing.

As described herein, activity, signals, expression, or function can be modulated (e.g., reduced, eliminated, or enhanced) using genome editing (e.g., upregulate, downregulate, overexpress, underexpress, express (e.g., transgenic expression), knock in, knock out, knockdown).

Processes for genome editing are well known; see e.g., Aldi 2018 Nature Communications 9 (1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage of gene/protein expression/signaling by genome editing can result in protection from autoimmune or inflammatory diseases.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications to target cells by the removal or addition of signals (e.g., activate (e.g., CRISPRa), upregulate, overexpress, downregulate).

For example, the methods as described herein can comprise a method for altering a target polynucleotide sequence in a cell comprising contacting the polynucleotide sequence with a clustered regularly interspaced short palindromic repeats-associated (Cas) protein.

### Gene Therapy and Genome Editing

Gene therapies can include inserting a functional gene with a viral vector. Gene therapies are rapidly advancing.

There has recently been an improved landscape for gene therapies. For example, in the first quarter of 2019, there were 372 ongoing gene therapy clinical trials (Alliance for Regenerative Medicine, May 9, 2019).

Any vector known in the art can be used. For example, the vector can be a viral vector selected from retrovirus, lentivirus, herpes, adenovirus, adeno-associated virus (AAV), rabies, Ebola, lentivirus, or hybrids thereof.

Gene therapy strategies.

| | Strategy |
|---|---|
| Viral Vectors | |
| Retroviruses | Retroviruses are RNA viruses transcribing |
| | their single-stranded |
| | genome into a double-stranded DNA copy, |
| | which can integrate into host chromosome |
| Adenoviruses (Ad) | Ad can transfect a variety of quiescent and |
| | proliferating |
| | cell types from various species and can |
| | mediate |
| | robust gene expression |
| Adeno-associated | Recombinant AAV vectors contain no viral |
| Viruses (AAV) | DNA and can carry ~4.7 kb of foreign |
| | transgenic material. They |
| | are replication defective and can replicate |
| | only while |
| | coinfecting with a helper virus |
| Non-viral vectors | |
| plasmid DNA | pDNA has many desired characteristics as a |
| (pDNA) | gene |
| | therapy vector; there are no limits on the size |
| | or genetic |
| | constitution of DNA, it is relatively |
| | inexpensive to supply, |
| | and unlike viruses, antibodies are not |
| | generated |
| | against DNA in normal individuals |
| RNAi | RNAi is a powerful tool for gene specific |
| | silencing that |
| | could be useful as an enzyme reduction |
| | therapy or |
| | means to promote read-through of a |
| | premature stop |
| | codon |

Gene therapy can allow for the constant delivery of the enzyme directly to target organs and eliminates the need for weekly infusions. Also, correction of a few cells could lead to the enzyme being secreted into the circulation and taken up by their neighboring cells (cross-correction), resulting in widespread correction of the biochemical defects. As such, the number of cells that must be modified with a gene transfer vector is relatively low.

Genetic modification can be performed either ex vivo or in vivo. The ex vivo strategy is based on the modification of cells in culture and transplantation of the modified cell into a patient. Cells that are most commonly considered therapeutic targets for monogenic diseases are stem cells. Advances in the collection and isolation of these cells from a variety of sources have promoted autologous gene therapy as a viable option.

The use of endonucleases for targeted genome editing can solve the limitations presented by the usual gene therapy protocols. These enzymes are custom molecular scissors, allowing cutting DNA into well-defined, perfectly specified pieces, in virtually all cell types. Moreover, they can be delivered to the cells by plasmids that transiently express the nucleases, or by transcribed RNA, avoiding the use of viruses.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc., may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided is a process of treating, preventing, or reversing intestinal conditions (including chronic intestinal conditions such as lactose intolerance, phenylketonuria, and inflammatory bowel disease) in a subject in need thereof via administration of a therapeutically effective amount of therapeutic-loaded OMVs, so as to deliver therapeutic compounds/enzymes directly to the gut microbiome.

Methods described herein are generally performed on a subject in need thereof. A subject in need of the therapeutic methods described herein can be a subject having, diagnosed with, suspected of having, or at risk for developing intestinal conditions. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of therapeutic-loaded OMVs is, for example, an amount that would cause the desired therapeutic effect in a subject while minimizing undesired side effects. In various embodiments, an effective amount of therapeutic-loaded OMVs described herein can substantially inhibit, slow the progress of, or limit the development of intestinal conditions (including chronic intestinal conditions).

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of therapeutic-loaded OMVs can be employed in pure form or, where such forms exist, in pharmaceutically acceptable salt form and with or without a pharmaceutically acceptable excipient. For example, the compounds of the present disclosure can be administered via an engineered Bacteroides thetaiotaomicron (Bt) strain as described herein, at a reasonable benefit/risk ratio applicable to any medical treatment, in a sufficient amount to deliver therapeutic compounds/enzymes directly to the gut microbiome.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes reversing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of therapeutic-loaded OMVs can occur as a single event or over a time course of treatment. For example, therapeutic-loaded OMVs can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for intestinal conditions (including chronic intestinal conditions).

Therapeutic-loaded OMVs can be administered simultaneously or sequentially with another agent, such as an antibiotic, an anti-inflammatory, or another agent. For example, therapeutic-loaded OMVs can be administered simultaneously with another agent, such as an antibiotic or an anti-inflammatory. Simultaneous administration can occur through administration of separate compositions, each containing one or more of therapeutic-loaded OMVs, an antibiotic, an anti-inflammatory, or another agent. Simultaneous administration can occur through administration of one composition containing two or more of therapeutic-loaded OMVs, an antibiotic, an anti-inflammatory, or another agent. Therapeutic-loaded OMVs can be administered sequentially with an antibiotic, an anti-inflammatory, or another agent. For example, therapeutic-loaded OMVs can be administered before or after administration of an antibiotic, an anti-inflammatory, or another agent.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general, a human equivalent dose (HED) in mg/kg can be calculated in accordance with the following formula (see e.g., Reagan-Shaw et al., FASEB J., 22 (3): 659-661, 2008, which is incorporated herein by reference):

$$HED ( mg / kg ) = Animal dose ( mg / kg ) \times ( Animal {K}_{m} / Human {K}_{m} )$$

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment, and the potency, stability, and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, the therapeutic agent (e.g., compound, composition, formulation, enzyme, etc.) may be administered in an amount from about 1 mg/kg to about 100 mg/kg, or about 1 mg/kg to about 50 mg/kg, or about 1 mg/kg to about 25 mg/kg, or about 1 mg/kg to about 15 mg/kg, or about 1 mg/kg to about 10 mg/kg, or about 1 mg/kg to about 5 mg/kg, or about 3 mg/kg. In some embodiments, a therapeutic agent may be administered in a range of about 1 mg/kg to about 200 mg/kg, or about 50 mg/kg to about 200 mg/kg, or about 50 mg/kg to about 100 mg/kg, or about 75 mg/kg to about 100 mg/kg, or about 100 mg/kg.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day.

In other non-limiting examples, a dose may also comprise from about 1 micro-gram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### Cell Therapy

Cells generated according to the methods described herein can be used in cell therapy. Cell therapy (also called cellular therapy, cell transplantation, or cytotherapy) can be a therapy in which viable cells are injected, grafted, or implanted into a patient in order to effectuate a medicinal effect or therapeutic benefit. For example, transplanting T-cells capable of fighting cancer cells via cell-mediated immunity can be used in the course of immunotherapy, grafting stem cells can be used to regenerate diseased tissues, or transplanting beta cells can be used to treat diabetes.

Stem cell and cell transplantation has gained significant interest by researchers as a potential new therapeutic strategy for a wide range of diseases, in particular for degenerative and immunogenic pathologies.

Allogeneic cell therapy or allogenic transplantation uses donor cells from a different subject than the recipient of the cells. A benefit of an allogeneic strategy is that unmatched allogenic cell therapies can form the basis of “off the shelf” products.

Autologous cell therapy or autologous transplantation uses cells that are derived from the subject's own tissues. It could also involve the isolation of matured cells from diseased tissues, to be later re-implanted at the same or neighboring tissues. A benefit of an autologous strategy is that there is limited concern for immunogenic responses or transplant rejection.

Xenogeneic cell therapies or xenotransplantation uses cells from another species. For example, pig derived cells can be transplanted into humans. Xenogeneic cell therapies can involve human cell transplantation into experimental animal models for assessment of efficacy and safety or enable xenogeneic strategies to humans as well.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10:0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

### Screening

Also provided are screening methods.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules database; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about-2 to about 4). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number among them). While these rules generally relate to oral absorption and are used to predict the bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to, reagents, compounds, strains, compositions, and formulations described herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754; Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1-Dual Membrane-Spanning Anti-Sigma Factors Regulate Vesiculation in Bacteroides Thetaiotaomicron

In Example 1, a high-throughput screen to identify components of the machinery involved in OMV biogenesis and its regulation in Bt was developed. A family of structurally unique Dual membrane-spanning anti-sigma factors (Dma) were identified and characterized and their role in modulating OMV biogenesis in Bt was investigated.

Bacteroidota are abundant members of the human gut microbiota that shape the enteric landscape by modulating host immunity and degrading dietary- and host-derived glycans. These processes are mediated in part by Outer Membrane Vesicles (OMVs). In Example 1, a high-throughput screen was developed to identify genes required for OMV biogenesis and its regulation in Bacteroides thetaiotaomicron (Bt). A family of Dual membrane-spanning anti-sigma factors (Dma) that control OMV biogenesis were identified. Molecular and multiomic analyses were conducted to demonstrate that deletion of Dma1, the founding member of the Dma family, modulates OMV production by controlling the activity of the ECF21 family sigma factor, Das1, and its downstream regulon. Dma1 has a previously uncharacterized domain organization that enables Dma1 to span both the inner and outer membrane of Bt. Phylogenetic analyses reveal that this common feature of the Dma family is restricted to the phylum Bacteroidota. The present disclosure provides mechanistic insights into the regulation of OMV biogenesis in human gut bacteria.

### Results

### Screening for Genes Involved in OMV Biogenesis.

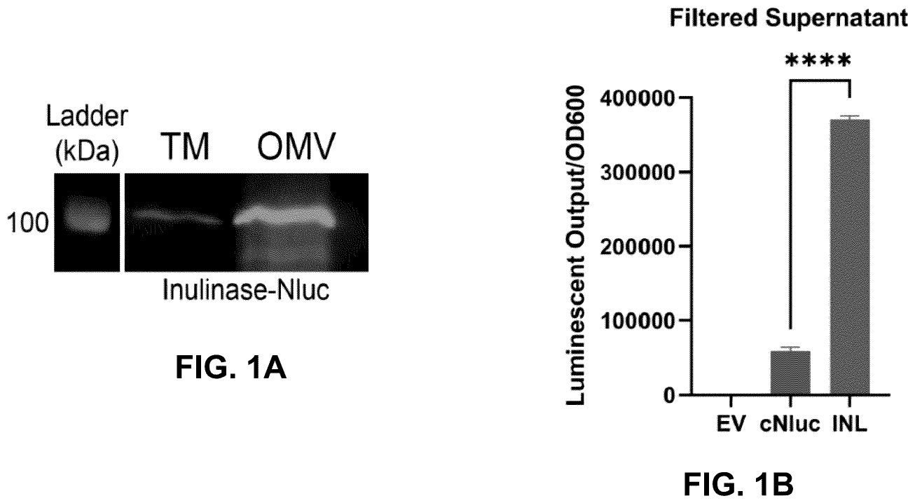

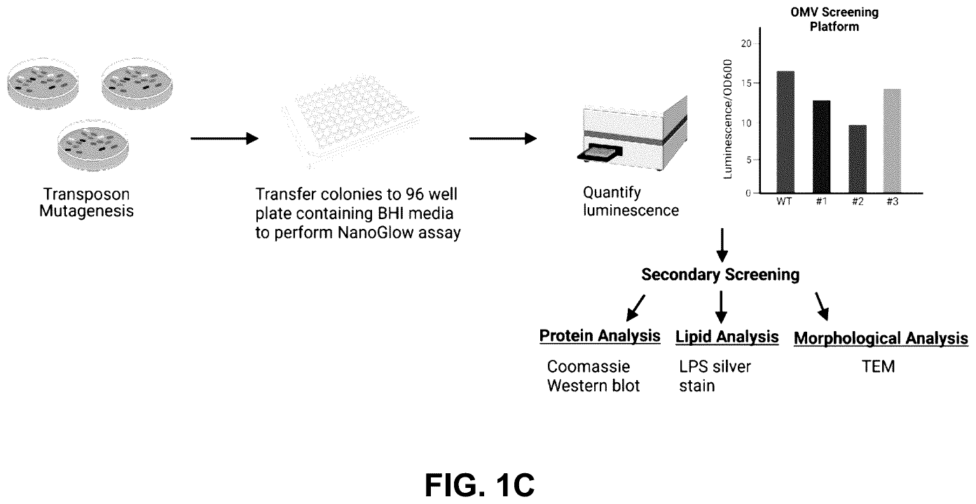

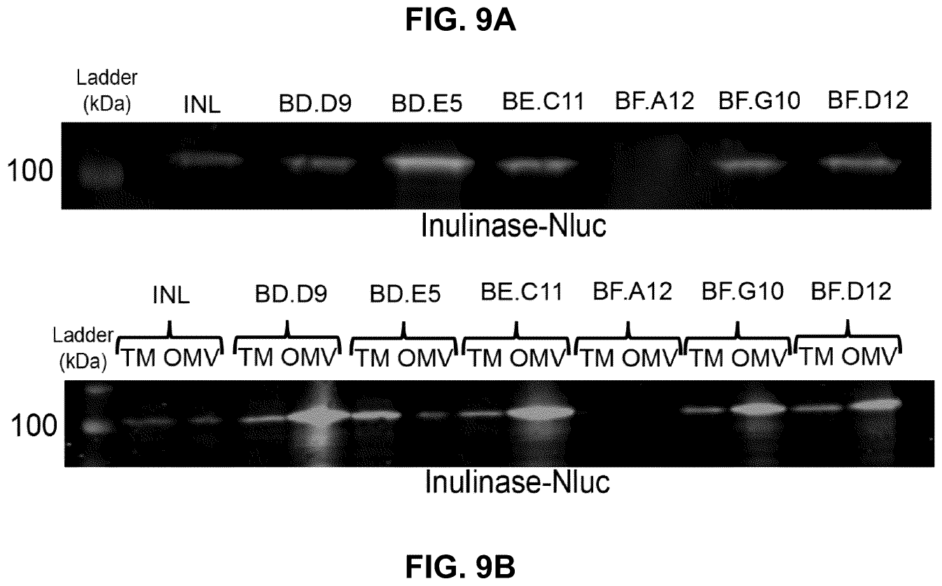

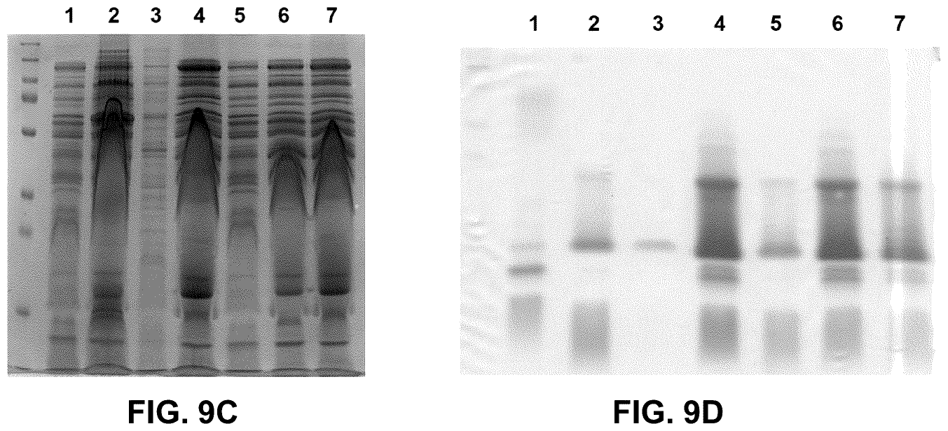

Vesiculation has been studied in gram-negative bacteria for ˜60 y; however, understanding of OMV biogenesis is still in its infancy. To identify bacterial proteins involved in OMV biogenesis in Bt, a high-throughput assay to quantify OMV production in vitro was developed. An OMV reporter was constructed consisting of Bacteroides ovatus inulinase (BACOVA_04502; INL), a protein previously shown to be enriched in OMVs, fused to Nanoluciferase (NLuc). Western blotting confirmed that the fusion protein was stable and almost exclusively present in OMVs (FIG. 1A). Filtered supernatants from cells constitutively expressing INL-NLuc exhibited significantly higher luminescent output than a lysis control strain expressing cytoplasmic NLuc (cNLuc) (FIG. 1B). This demonstrates that luminescence in culture supernatants can be used as an easily quantifiable proxy for OMV production. Next, a transposon library was created in the strain constitutively expressing the OMV reporter, INL-NLuc. Since the reporter is almost exclusively trafficked to OMVs, it was anticipated that mutants displaying abnormal levels of NLuc activity in their supernatants corresponded to abnormal levels of OMV production. The strategy employed for the screening is described in FIG. 1C. Approximately 5,300 colonies were screened and detected several mutants displaying abnormally high or low levels of NLuc activity were detected (FIG. 9). Secondary screening, which consisted of TEM, western blotting, and LPS and protein analysis, was performed to validate potential candidates (FIG. 9). The genomes of mutants displaying atypical levels of vesiculation were sequenced to identify the transposon insertion sites. Altogether, these analyses identified several hyper- and hypovesiculating strains, summarized in Table 1 below.

TABLE 1
List of candidate genes identified during OMV Screen.

| Candidate | Insertion Site(s) | Gene(s) Interrupted | Gene Annotations |
|---|---|---|---|
| C.A7 | 935,471 | BT_0753 | Anti-sigma factor |
| C.A9 | 458,218; 1,402,418 | BT_0372; BT_1115 | Aldose 1-epimerase; Aldo/keto reductase |
| C.B9 | 4,377,534 | BT_3397 | Pyruvate ferredoxin oxidoreductase |
| G.A1 | 6,138,309 | N/A | Insertion in INL-Nluc expression vector |
| N.D2 | 2,722,830 | N/A | Insertion in intergene region between |
| | | | BT_2164-65 |
| S.C3 | 3,456,177 | BT_2786 | 30S ribosomal protein s15 |
| V.E10 | 4,545,446; 5,611,550 | BT_3519; BT_4261 | SusC-like protein; DUF4847 domain- |
| | | | containing protein |
| V.F3 | 2,803,639 | BT_2235 | Hypothetical protein |
| W.H11 | 6,138,327 | N/A | Insertion in INL-Nluc expression vector |
| X.H3 | 5,611,256 | BT_4261 | DUF4847 domain-containing protein |
| AA.A11 | 4,410,790; 4,545,446 | BT_3422; BT_3519 | alpha-2-macroglobulin; SusC-like protein |
| AK.D1 | 3,115,604 | BT_2493 | ROK family protein |
| AL.B5 | 3,115,511 | BT_2493 | ROK family protein |
| AL.G3 | N/A | N/A | |
| AM.C1 | 3,116,101 | BT_2493 | ROK family protein |
| AT.E8 | N/A | N/A | |
| AT.F1 | 4,820,482 | BT_3709 | Elongation Factor P |
| AT.F10 | 4,820,482 | BT_3709 | Elongation Factor P |
| AX.G6 | 6,196,151 | BT_4721 | Outer membrane beta-barrel domain- |
| | | | containing protein |
| BA.D10 | 4,820,482 | BT_3709 | Elongation Factor P |
| BB.E11 | 3,115,663 | BT_2493 | ROK family protein |
| BD.D9 | 374,098; 4,155,947 | BT_0311; BT_3255 | 2-oxo acid dehydrogenase; N6-adenine- |
| | | | specific DNA methylase |
| BD.E5 | 4,304,745 | BT_3341 | SbsA Ig-like domain-containing protein |
| BE.C11 | 6,196,148 | BT_4721 | Outer membrane beta-barrel domain- |
| | | | containing protein |
| BF.A12 | 6,138,331 | N/A | Insertion in INL-Nluc expression vector |
| BF.D12 | 6,196,613 | BT_4721 | Outer membrane beta-barrel domain- |
| | | | containing protein |
| BF.G10 | 6,196,617 | BT_4721 | Outer membrane beta-barrel domain- |
| | | | containing protein |

### Mutation of Dma1 (BT_4721) Leads to Hypervesiculation.

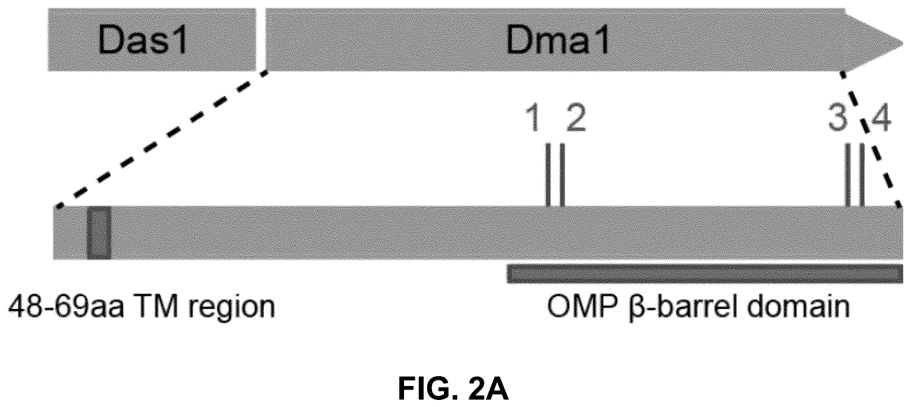

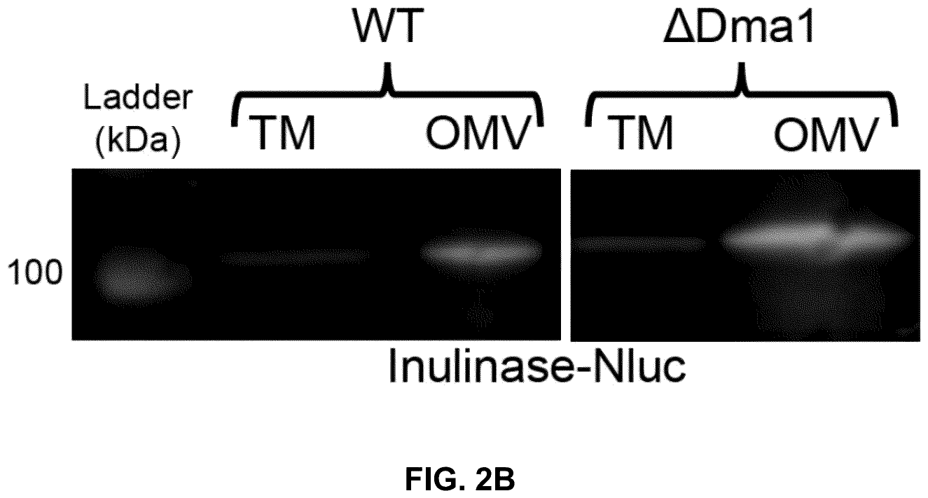

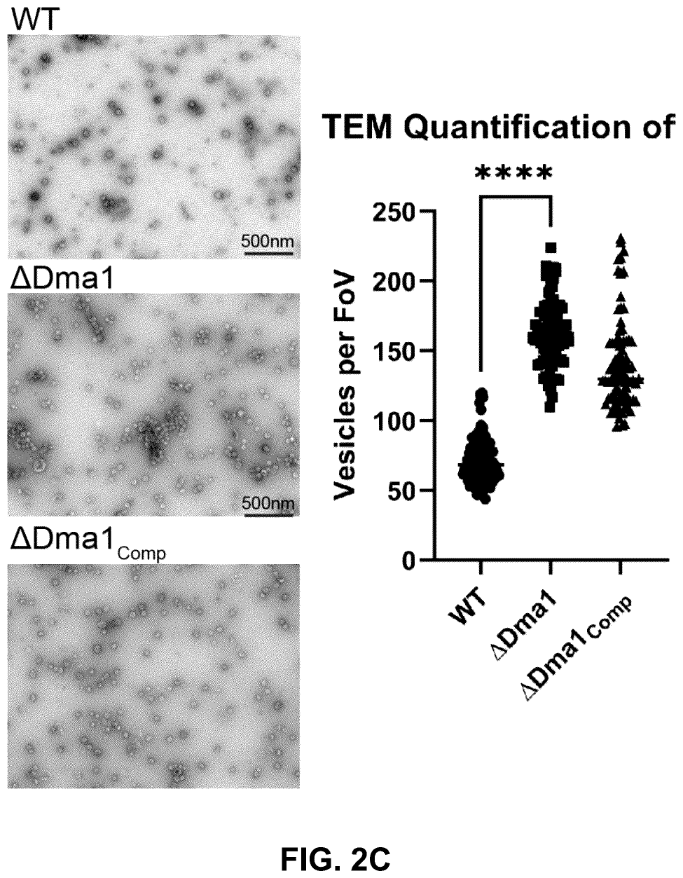

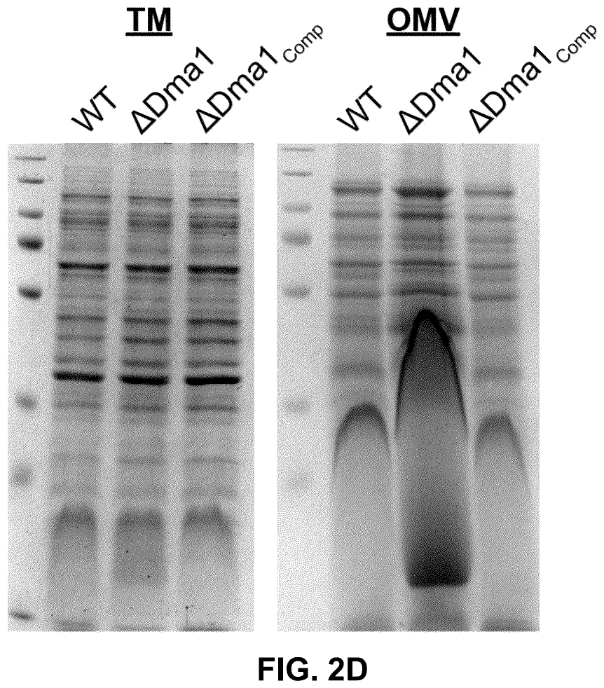

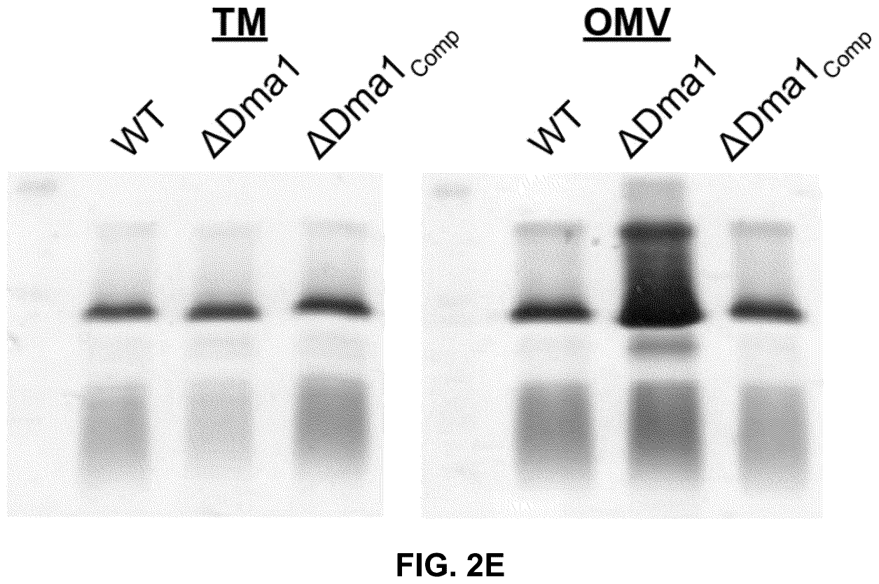

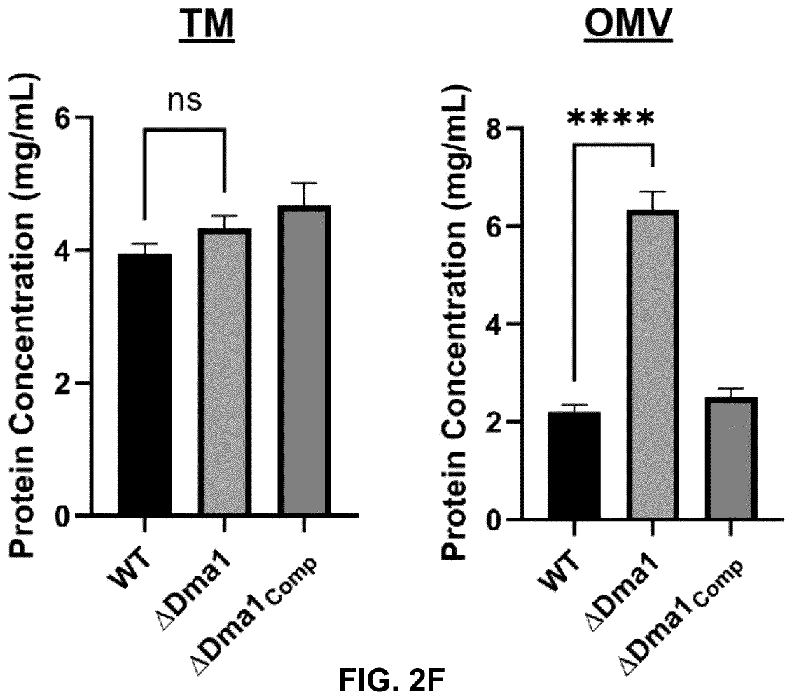

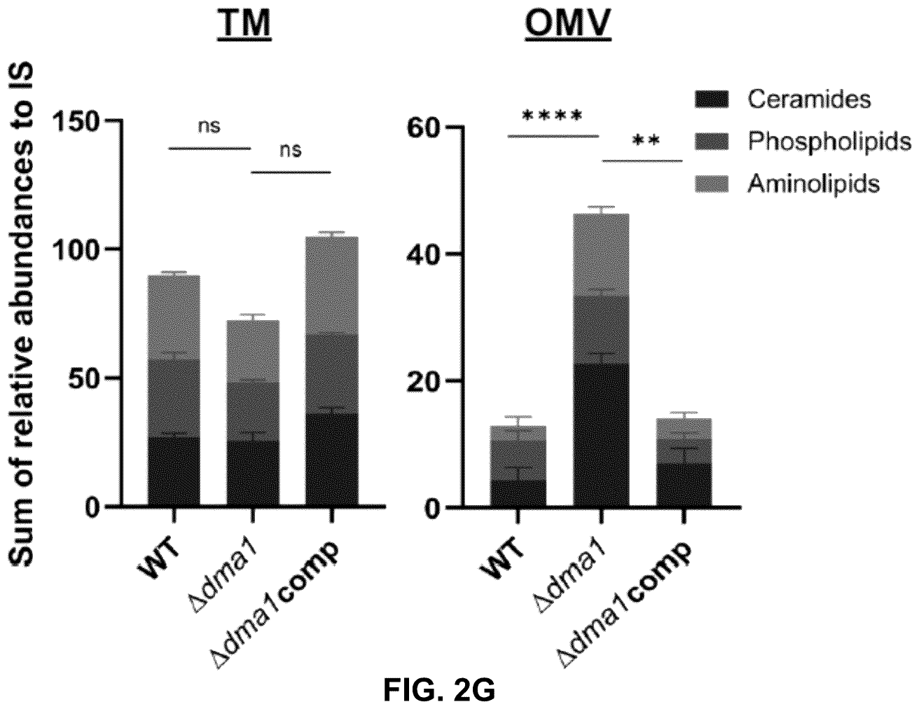

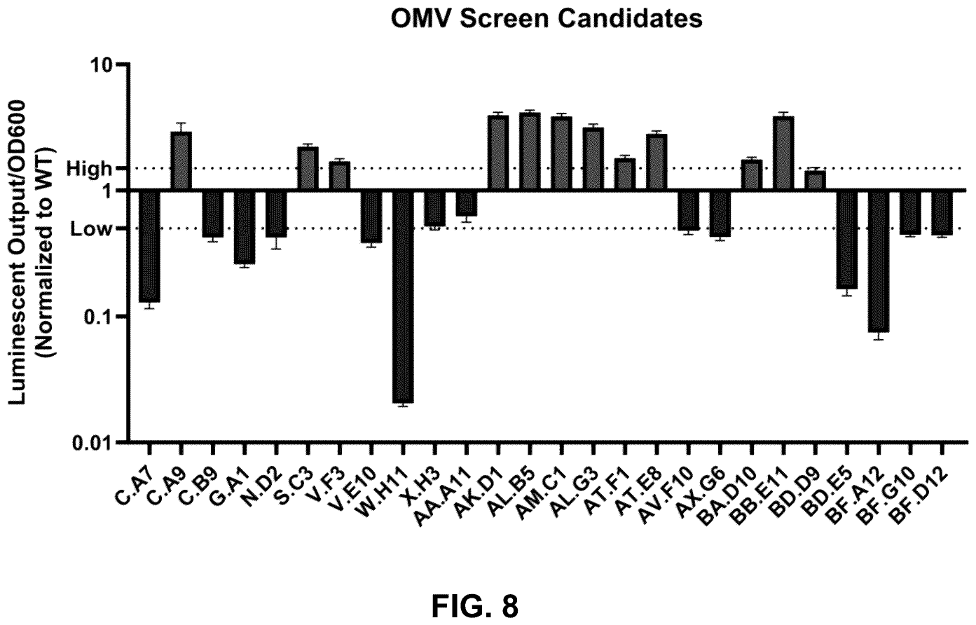

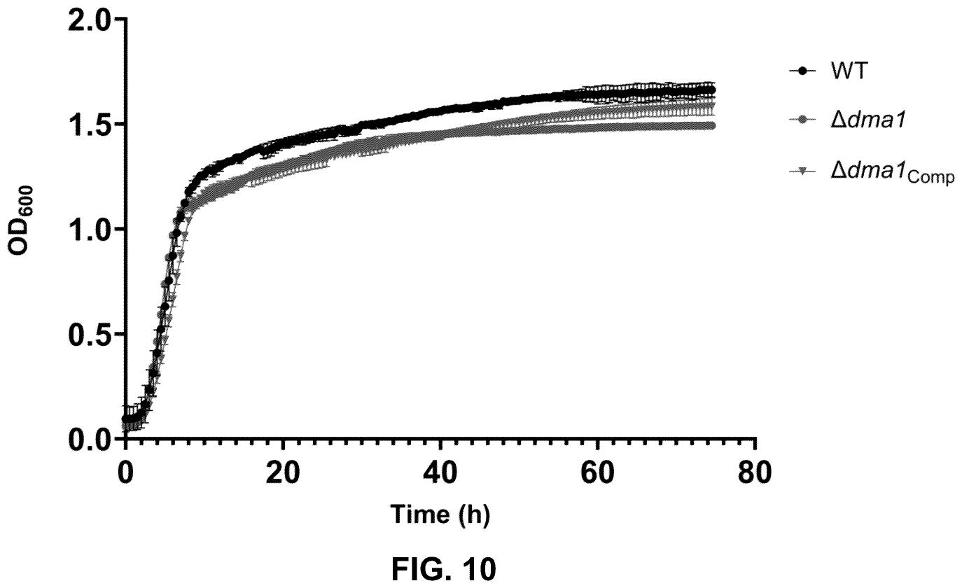

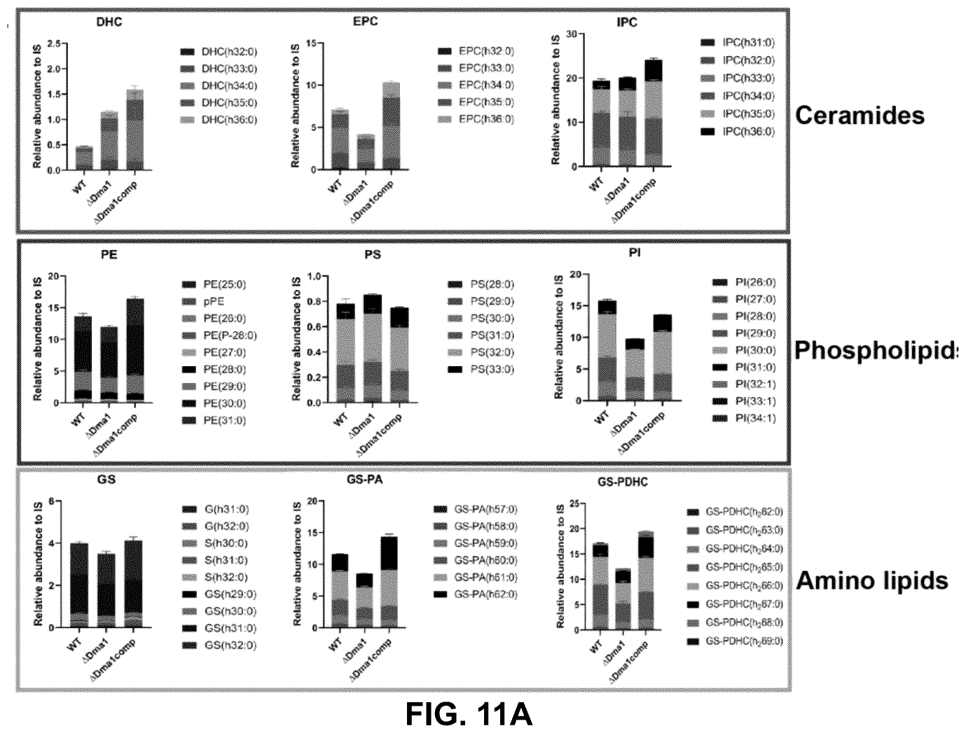

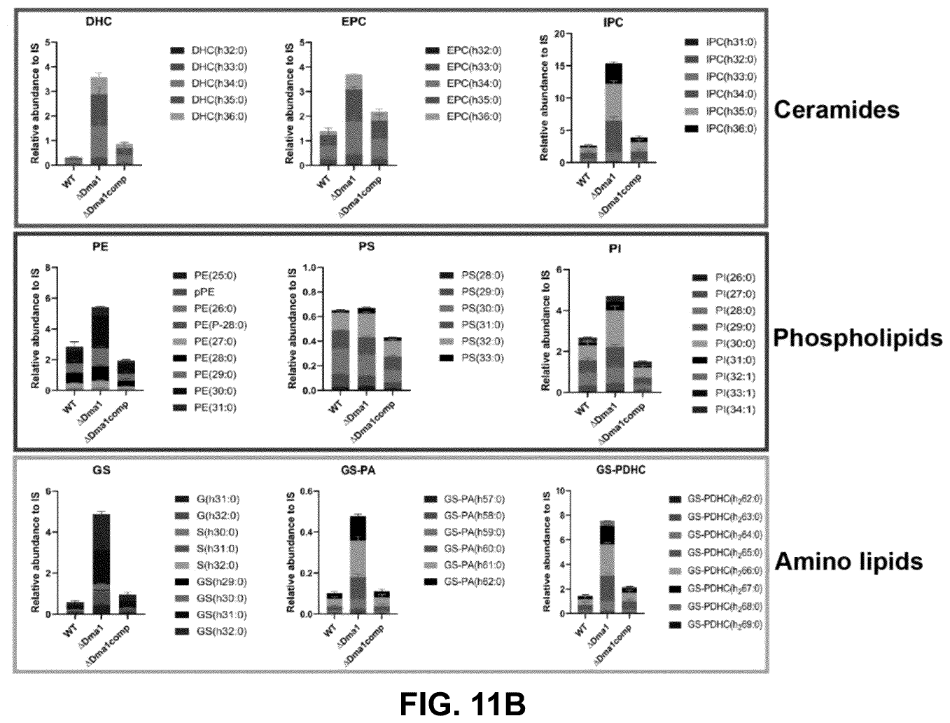

Genomic sequencing of potential candidates revealed four independent mutants containing transposon insertions in the gene BT_4721, which was renamed Dma1 (FIG. 2A). In the initial screening, these four strains appeared to display a hypovesiculating phenotype (FIG. 8 and Table 1). However, western blots showed that these mutants had increased abundance of INL-NLuc in the OMV fraction (FIG. 2B). To further analyze the role of Dma1, a Dma1 deletion mutant (Δdma1) and its corresponding complemented strain (Δdma1Comp) were generated. Growth curve s confirmed that the fitness of Δdma1 is not attenuated in this context (FIG. 10). Next, OMVs were isolated and quantified by TEM. This analysis confirmed the western blot data showing that Δdma1 produces significantly more OMVs than the WT (FIG. 2C). Admittedly, the ability to accurately quantify OMVs using TEM and manual counting is limited. However, since Bacteroides OMVs contain lipopolysaccharide (LPS), membrane lipids (phospholipids, sphingolipids, and amino lipids), and protein cargo, it was aimed to confirm the phenotype of Δdma1 biochemically by quantifying the relative amount of these components in total membrane (inner and outer; TM) and OMV preparations from WT, Δdma1, and Δdma1 Comp. First, their respective protein content was analyzed by SDS-PAGE. Surprisingly, the electrophoretic profile of the OMV fraction from Δdma1 exhibited a significant distortion in the low molecular weight region of the gel, which was reverted by complementation (FIG. 2D). The distortion appeared to be due to the presence of high amounts of LPS in the samples due to Δdma1 hypervesiculation. Analysis of LPS from TM and OMV fractions via SDS-PAGE followed by silver stain confirmed that Δdma1 secretes significantly more LPS than the wild-type and complemented strains (FIG. 2E). The OMV fraction of Δdma1 also contained more proteins than the WT and complemented strains (FIG. 2F). Finally, comparative lipidomics was performed on TM and OMV fractions from WT, Δdma1, and Δdma1Comp. While the lipid composition for both fractions remained relatively unperturbed, a general increase in secreted phospho, sphingo-, and amino lipid content was detected in the Δdma1 OMVs (FIG. 2G and FIG. 11). Altogether, the findings demonstrate that Δdma1 produces significantly more OMVs than its parental strain.

### Deletion of Dma1 Modulates OMV Production but not Composition.

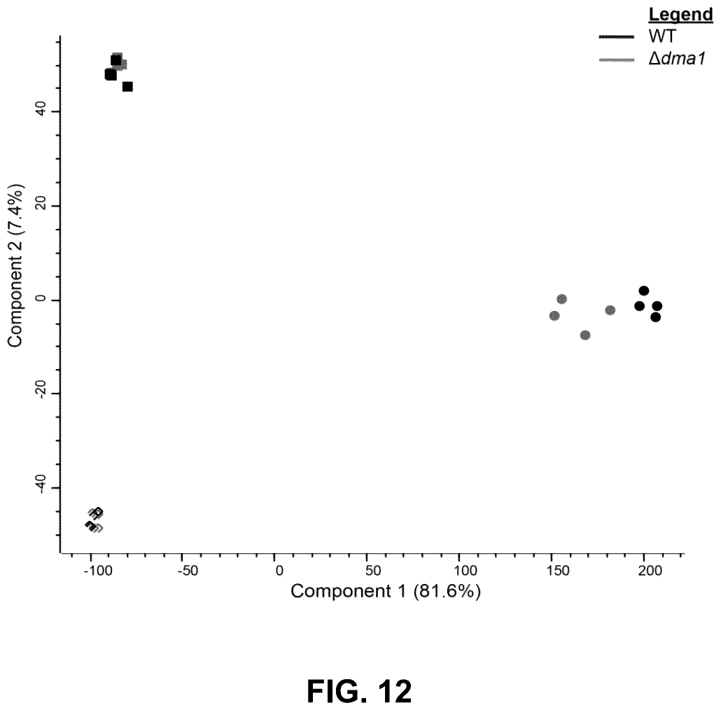

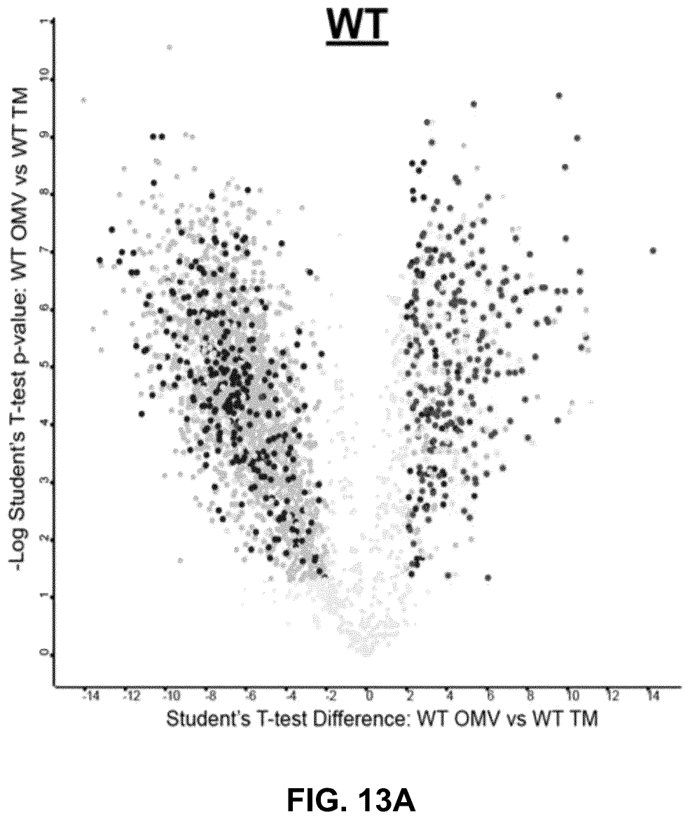

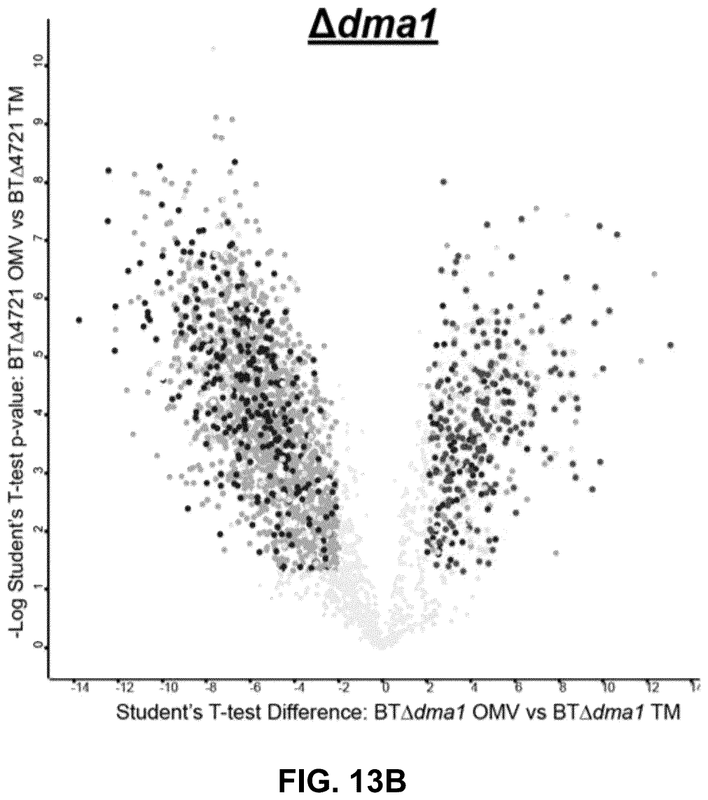

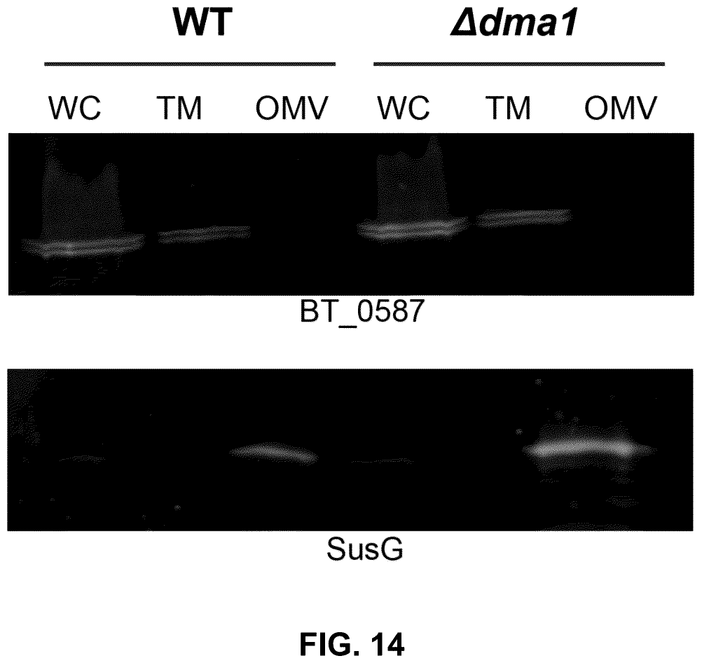

To demonstrate that the hypervesiculation phenotype of Δdma1 is due to an increase in the production of bona fide OMVs and not the result of a generalized destabilization of the outer membrane (OM), comparative proteomic analyses of TM and OMV fractions from the WT and Δdma1 strains was performed. A principal component analysis shows that the TM and OMV fractions of the WT and mutant contain similar protein content (FIG. 12). Bacteroides OMVs are enriched with LES motif containing lipoproteins, while other OM proteins are typically excluded. Volcano plots show that similar to the WT, the OMVs of Δdma1 are primarily enriched with lipoproteins containing the LES motif, while porins and other proteins were retained predominantly in the bacterial membranes (FIG. 13). Thus, OMV cargo selection is maintained in the Δdma1, which rules out that the increased amount of OMVs is the result of increased cell lysis or membrane instability, which would result in the presence of cytosolic and inner membrane (IM) proteins in the OMV proteome. Further supporting this conclusion, western blots show that the partitioning of the OMV protein SusG and the OM protein BT_0587 is not affected in Δdma1 (FIG. 14).

### Extracytoplasmic Function (ECF) Sigma Factor, Das1 (BT_4720), is Required for Dma1-Mediated Hypervesiculation.

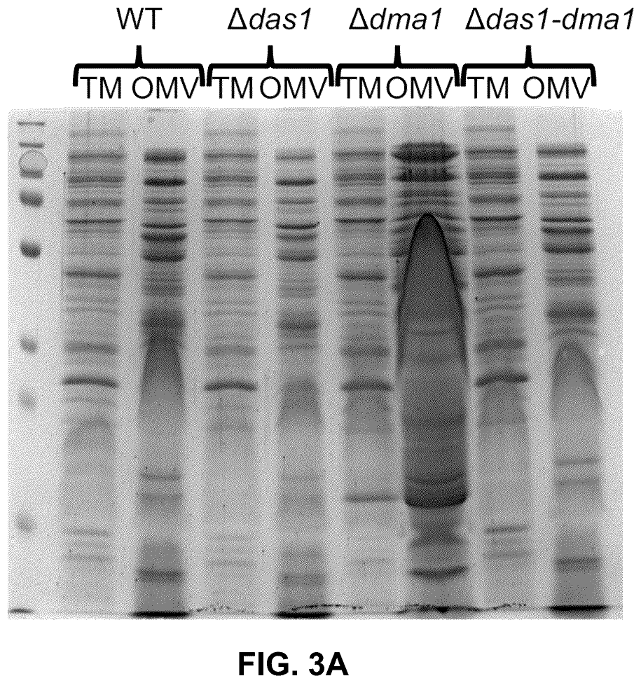

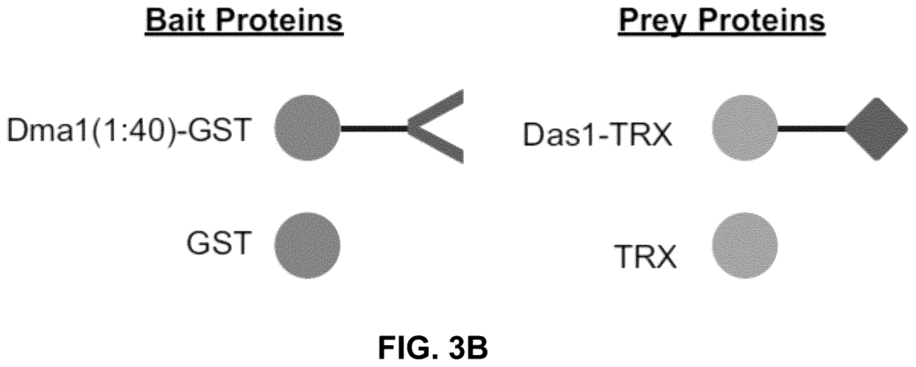

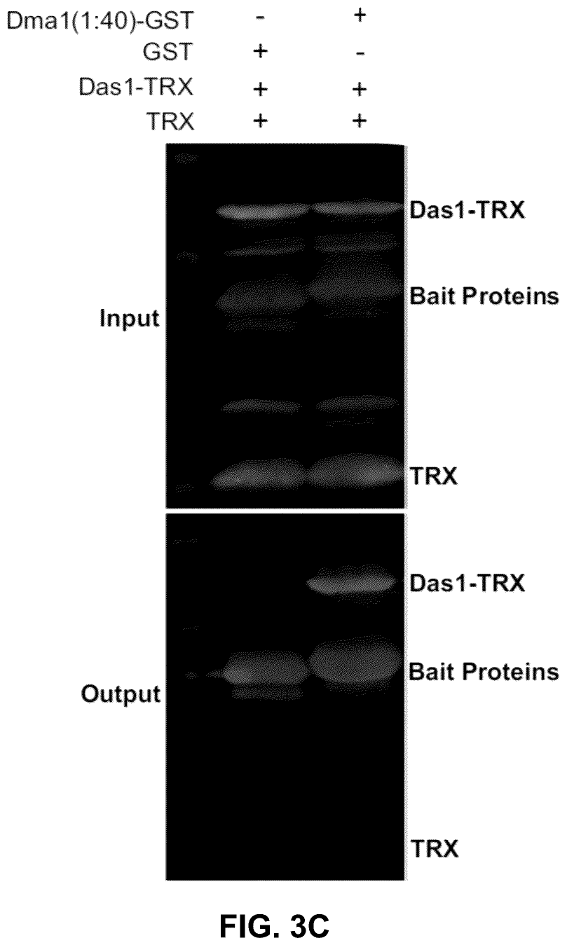

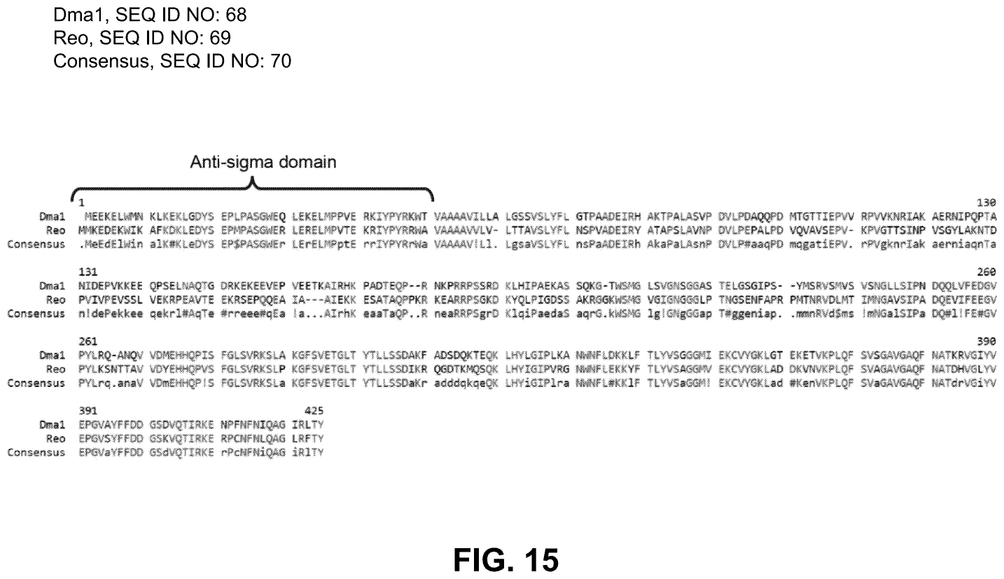

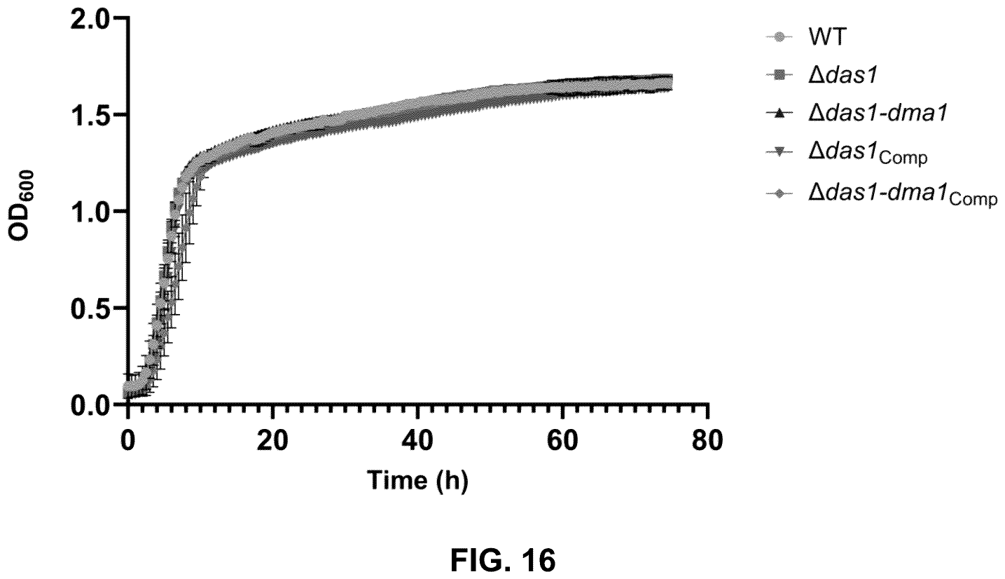

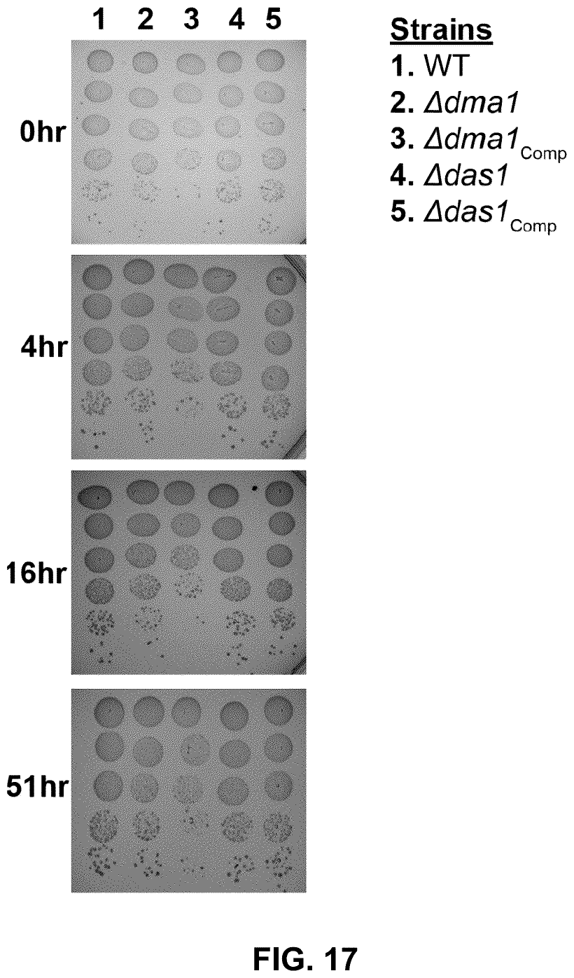

Dma1 is encoded in an operon with the ECF21 family sigma factor, BT_4720 (Das1, for Dma-associated sigma factor 1. ECF-type sigma factors are a family of transcriptional regulators that modulate gene expression in response to extracytoplasmic signals. These are typically encoded adjacent to their cognate anti-sigma factor, which negatively regulates the activity of the sigma factor. ECF21 family sigma factors are found solely among Bacteroidota, but very little is known regarding their function. An ortholog of Dma1 in Bf, Reo, was shown to control the activity of its cognate sigma factor, ecfO, by sequestering it at the IM via its N-terminal region in the cytoplasm. There is strong consensus between the amino acid sequences from Reo and Dma1 (FIG. 15), which suggests that Dma1 possesses a cytoplasmic domain capable of directly regulating the activity of Das1. To provide support for the interaction between Dma1 and Das1, Das1 was mutated in the Δdma1 background (Δdas1-dma1). A single deletion mutant lacking Das1 (Δdas1) was included as control. Growth curves show that the fitness of these mutants is not impacted in vitro (FIG. 16). By employing SDS-PAGE, it was determined that removal of Das1 reverts the distortion in the electrophoretic pattern seen for Δdma1 OMVs (FIG. 3A), indicating that the hypervesiculation observed in Δdma1 requires Das1. Next, targeted pulldowns were performed to determine whether Dma1 and Das1 physically interact. For this, a chimeric protein was engineered containing the N-terminal 40 amino acids of Dma1 fused to Glutathione-S transferase (GST), Dma1 (1:40)-GST, as the bait protein. The prey protein consisted of Das1 fused to thioredoxin (TRX), Das1-TRX. Controls expressing GST and TRX alone were included (FIG. 3B). Each protein was individually expressed in Escherichia coli BL21. Lysates from strains expressing each bait protein were mixed with lysates containing the prey proteins and subjected to affinity chromatography by employing a column containing anti-GST resin. The results demonstrate that Dma1 (1:40)-GST specifically interacts with Das1-TRX, indicating that the N-terminal region of Dma1 is sufficient to sequester Das1 (FIG. 3C). Together, these findings demonstrate that Das1 and Dma1 form a sigma/anti-sigma pair and that the unregulated activity of Das1 is responsible for hypervesiculation observed in Δdma1. A previous study has shown that deletion of Reo, in Bf, increases fitness in response to oxidative stressors, while deletion of ecfO renders them more susceptible. The fitness of WT, Δdas1, Δdma1, and their corresponding complemented strains in response to prolonged aerobic stress was assessed no changes between the mutants and the wild type were observed (FIG. 17). This suggests that Dma1 and Reo control different responses in Bt and Bf, respectively.

### NigD1, Part of the Dma1-Das1 Regulon, is Necessary for Hypervesiculation.

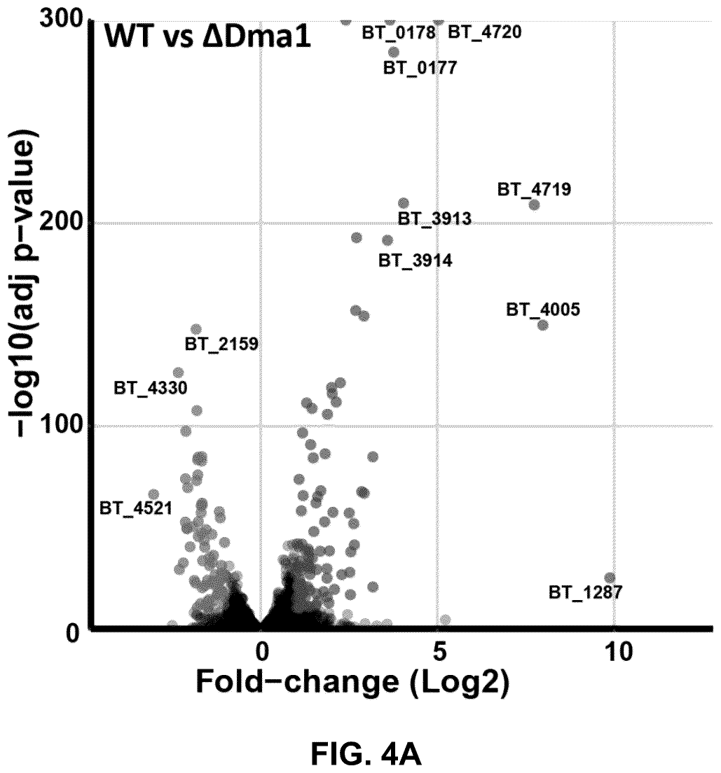

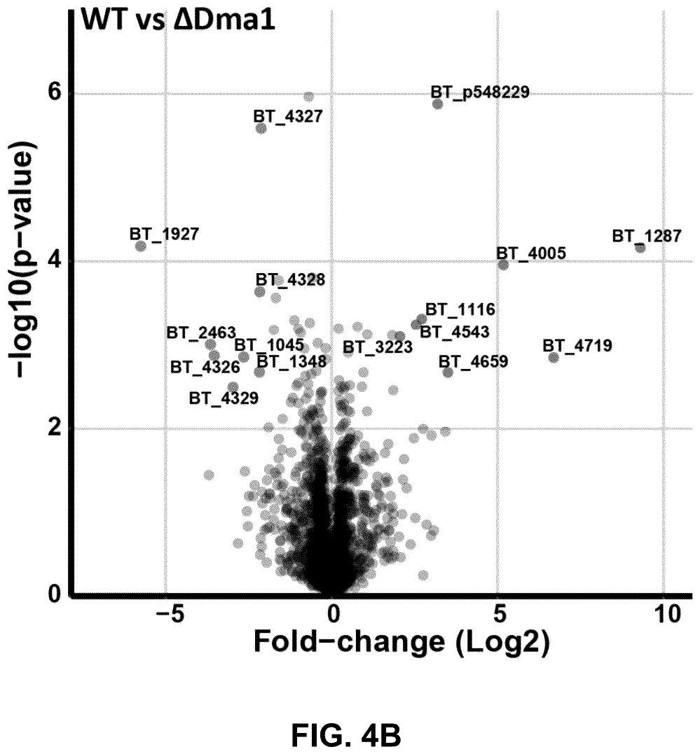

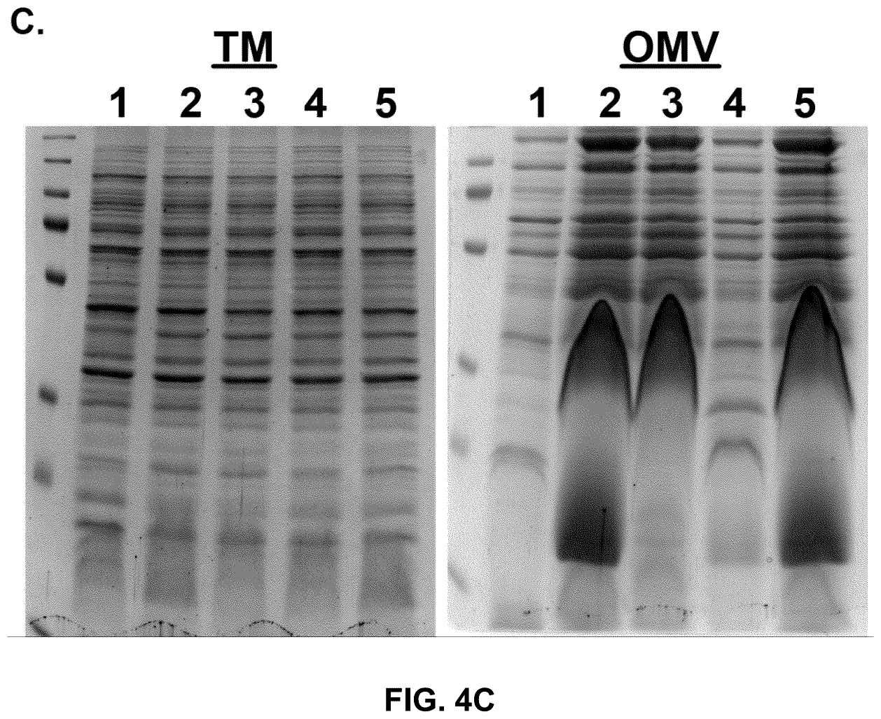

To understand how Dma1 controls vesiculation, RNA sequencing was performed to compare transcriptomes of the wild-type and Δdma1 strains. It was found that three genes, BT_1287, NigD1 (BT_4005), and NigD2 (BT_4719), were dramatically up-regulated in the Δdma1 mutant (FIG. 4A and Table 2). There were other genes differentially expressed (Table 2), but their changes were much less pronounced.

TABLE 2
List of the Δdma1 Top 25 most upregulated and
downregulated genes as determined by RNA sequencing.

| | | | | |
|---|---|---|---|---|
| Gene (New | Gene (Old | | | |
| Locus Tag) | Locus Tag) | Log₂FC | Padj | Proposed Function |

RNA Sequencing- Δdma1 Top 25 Most Upregulated Genes

| | | | | |
|---|---|---|---|---|
| BT_RS06495 | BT_1287 | 9.879633157 | 7.34E−26 | DUF4840 domain-containing |
| | | | | protein |
| BT_RS20210 | BT_4005 | 7.986054337 | 2.34E−150 | NigD-like protein |
| BT_RS23775 | BT_4719 | 7.743518851 | 1.26E−209 | NigD-like protein |
| BT_RS16205 | NO | 5.229630823 | 3.24E−05 | Smalltalk protein |
| BT_RS23780 | BT_4720 | 5.034076276 | 0 | Sigma-70 family RNA polymerase |
| | | | | sigma factor |
| BT_RS19750 | BT_3913 | 4.044393237 | 1.72E−210 | DUF5034 domain-containing |
| | | | | protein |
| BT_RS00870 | BT_0177 | 3.767930416 | 6.95E−285 | NigD-like protein |
| BT_RS00875 | BT_0178 | 3.662030315 | 0 | YIP1 family protein |
| BT_RS19755 | BT_3914 | 3.590716188 | 3.32E−192 | Hypothetical protein |
| BT_RS11715 | BT_2315 | 3.579969558 | 0.0040536 | Hypothetical protein |
| BT_RS24670 | NO | 3.287601612 | 0.02828646 | Hypothetical protein |
| BT_RS05220 | BT_1038 | 3.178373436 | 2.30E−21 | endo-beta-N- |
| | | | | acetylglucosaminidase family |
| | | | | protein |
| BT_RS20385 | BT_4039 | 3.177706063 | 1.72E−85 | TonB-dependent receptor |
| BT_RS23805 | BT_4725 | 2.955721786 | 0.00240863 | RagB/SusD family nutrient uptake |
| | | | | outer membrane protein |
| BT_RS05230 | BT_1040 | 2.931638602 | 1.49E−67 | SusC/RagA family TonB-linked |
| | | | | outer membrane protein |
| BT_RS22810 | BT_4523 | 2.926352615 | 7.39E−155 | Type I restriction endonuclease |
| | | | | EcoR124II |
| BT_RS14080 | BT_2779 | 2.860410832 | 2.41E−68 | Class I SAM-dependent |
| | | | | methyltransferase |
| BT_R$10570 | BT_2086 | 2.691908858 | 1.37E−157 | Linear amide C-N hydrolase |
| BT_RS05210 | BT_1036 | 2.653696133 | 4.33E−42 | DUF1735 domain-containing |
| | | | | protein |
| BT_RS22805 | BT_4522 | 2.637320216 | 1.54E−52 | Type I restriction endonuclease |
| BT_RS05225 | BT_1039 | 2.5531964 | 1.13E−38 | SusD/RagB family nutrient-binding |
| | | | | outer membrane lipoprotein |
| BT_RS22645 | BT_4490 | 2.541660806 | 1.52E−17 | DUF5025 domain-containing |
| | | | | protein |
| BT_RS05215 | BT_1037 | 2.524779245 | 1.45E−28 | DUF 1735 and LamG domain- |
| | | | | containing protein |
| BT_RS12945 | BT_2560 | 2.50963332 | 8.48E−58 | TonB-dependent receptor |
| BT_RS14845 | NO | 2.460763495 | 9.28E−08 | Smalltalk protein |

RNA Sequencing- Δdma1 Top 25 Most Downregulated Genes

| | | | | |
|---|---|---|---|---|
| BT_RS22800 | BT_4521 | −3.0243084 | 5.47E−67 | Tyrosine-type |
| | | | | recombinase/integrase |
| BT_RS15310 | BT_3017 | −2.497832498 | 0.01664662 | Acid phosphatase |
| BT_RS21860 | BT_4330 | −2.326456539 | 5.53E−127 | Nucleoside permease |
| BT_RS09755 | BT 1926 | −2.295427189 | 5.78E−30 | OmpA/MotB domain protein |
| BT_RS18675 | BT_3704 | −2.184786525 | 3.07E−33 | Glycoside hydrolase family 13 |
| | | | | protein |
| BT_RS04750 | BT_0943 | −2.127262731 | 2.26E−53 | Penicillin-binding protein 2B |
| | | | | (PBP-2B) |
| BT_RS13245 | BT_2619 | −2.123420723 | 1.05E−74 | Histidine kinase |
| BT_RS21840 | BT_4326 | −2.112073399 | 4.79E−98 | ATP-binding cassette domain- |
| | | | | containing protein |
| BT_RS16435 | BT_3244 | −2.081737712 | 3.53E−50 | BACON domain-containing |
| | | | | protein |
| BT_RS04760 | BT_0945 | −2.078233418 | 3.80E−50 | Hypothetical protein |
| BT_RS14350 | BT_2830 | −2.060239218 | 2.68E−70 | HU family DNA-binding protein |
| BT_RS09760 | BT_1927 | −1.993238565 | 3.54E−41 | Hypothetical protein |
| BT_RS16430 | BT_3243 | −1.909968855 | 4.73E−51 | DUF4302 domain-containing |
| | | | | protein |
| BT_RS13240 | BT_2618 | −1.881918801 | 9.75E−09 | Two-component system |
| | | | | response regulator |
| BT_RS20375 | BT_4037 | −1.872169035 | 1.38E−24 | Hypothetical protein |
| BT_RS15060 | BT_2972 | −1.835815403 | 2.91E−23 | Class I SAM-dependent |
| | | | | methyltransferase |
| BT_RS10930 | BT_2159 | −1.822844005 | 2.25E−148 | Gfo/Idh/MocA family |
| | | | | oxidoreductase |
| BT_RS21835 | BT_4325 | −1.809901243 | 8.80E−74 | ABC transporter permease |
| BT_RS08875 | BT_1751 | −1.805310189 | 2.07E−07 | Glycine betaine/L-proline ABC |
| | | | | transporter ATP-binding protein |
| BT_RS21845 | BT_4327 | −1.801115206 | 2.77E−108 | DUF4836 family protein |
| BT_RS13230 | BT_2615 | −1.793091582 | 1.50E−07 | Group Il intron reverse |
| | | | | transcriptase/maturase |
| BT_RS04755 | BT_0944 | −1.791690553 | 3.24E−11 | Hypothetical protein |
| BT_RS13260 | BT_2622 | −1.780022703 | 5.06E−84 | Alpha-glucuronidase |
| BT_RS21850 | BT_4328 | −1.774306654 | 1.52E−76 | 16S rRNA (uracil(1498)-N(3))- |
| | | | | methyltransferase |
| BT_RS10860 | BT_2145 | −1.764647845 | 2.52E−85 | Adenosylcobalamin-dependent |
| | | | | ribonucleoside-diphosphate |
| | | | | reductase |

Comparative proteomic analyses of WT and Δdma1 identified the same three proteins as the most differentially expressed proteins between these strains (FIG. 4B and Table 3).

TABLE 3
List of the Δdma1 Top 25 most upregulated and downregulated
genes as determined by comparative proteomics.

| | | | | |
|---|---|---|---|---|
| Protein ID | Gene | Fold Change | −Log(P-value) | Proposed Function |

Comparative Proteomics- Δdma1 Top 25 Most Upregulated Genes

| | | | | |
|---|---|---|---|---|
| Q8A887 | BT_1287 | 9.30394 | 4.16088 | DUF4840 domain-containing |
| | | | | protein |
| Q89YL2 | BT_4719 | 6.68855 | 2.84572 | NigD-like protein |
| Q8A0L6 | BT_4005 | 5.17626 | 3.95591 | NigD-like protein |
| Q89YS2 | BT_4659 | 3.50177 | 2.6692 | SusD homolog |
| Q8AB86 | BT_0224 | 3.42438 | 1.96457 | Lipocalin-like protein |
| Q8ABW9 | BT_p548229 | 3.19438 | 5.8735 | Fimbrillin family protein |
| Q8A2W6 | BT_3189 | 3.012 | 1.91681 | Unknown |
| Q8ABX1 | BT_p548227 | 2.75674 | 1.99822 | DUF3575 domain-containing |
| | | | | protein |
| Q8A8Q1 | BT_1116 | 2.7145 | 3.31 | PPC domain-containing |
| | | | | protein |
| Q89Z34 | BT_4543 | 2.54599 | 3.24113 | Putative type I restriction |
| | | | | enzyme specificity protein |
| Q8A174 | BT_3792 | 2.47739 | 1.88394 | alpha-1,6-mannanase |
| Q8A0W2 | BT_3909 | 2.1813 | 1.63533 | Unknown |
| Q8A2T3 | BT_3222 | 2.146 | 1.39245 | DUF4848 domain-containing |
| | | | | protein |
| Q8A2T2 | BT_3223 | 2.05685 | 3.09966 | OMP_b-brl domain-containing |
| | | | | protein |
| Q8A6W1 | BT_1765 | 1.85376 | 2.45838 | Levanase (2,6-beta-D- |
| | | | | fructofuranosidase) |
| Q8A3A2 | BT_3052 | 1.82297 | 3.1166 | AraC family transcriptional |
| | | | | regulator |
| Q8A817 | BT_1180 | 1.70579 | 1.8163 | Glycoside transferase family 4 |
| Q8A0E0 | BT_4081 | 1.69799 | 1.36892 | SusC homolog |
| Q8A113 | BT_3858 | 1.64563 | 1.41392 | Alpha-1,2-mannosidase |
| Q8A9K1 | BT_0814 | 1.60821 | 1.70922 | BamA/TamA family outer |
| | | | | membrane protein |
| Q8AB18 | BT_0294 | 1.60238 | 1.35371 | Carboxypeptidase regulatory- |
| | | | | like domain-containing protein |
| Q8A5R5 | BT_2173 | 1.38299 | 1.42681 | SusD homolog |
| Q8A151 | BT_3816 | 1.33909 | 1.3211 | Penicillin-binding protein 2 |
| | | | | (PBP-2) |
| Q8AA46 | BT_0619 | 1.27055 | 1.6609 | Ion-translocating |
| | | | | oxidoreductase complex |
| | | | | subunit D, rnfD |
| Q8A109 | BT_3862 | 1.18718 | 1.70627 | Endo-alpha-mannosidase |

Comparative Proteomics- Δdma1 Top 25 Most Downregulated Genes

| | | | | |
|---|---|---|---|---|
| Q8A6F7 | BT_1927 | −5.75443 | 4.17785 | Unknown |
| Q8ABI4 | BT_0126 | −3.70228 | 1.44507 | Six-hairpin glycosidase |
| Q8A4Y4 | BT_2463 | −3.65105 | 3.00365 | RNA polymerase ECF- |
| | | | | type sigma factor |
| Q89ZQ0 | BT_4326 | −3.53782 | 2.86951 | ABC transporter ATP- |
| | | | | binding protein |
| Q89ZP7 | BT_4329 | −2.97173 | 2.49205 | BFN domain-containing |
| | | | | protein |
| Q8A8X2 | BT_1045 | −2.64843 | 2.85322 | Concanavalin A-like |
| | | | | lectin/glucanase |
| Q8A7D7 | BT_1587 | −2.61663 | 1.4901 | GCN5-related N- |
| | | | | acetyltransferase |
| Q8AAE0 | BT_0525 | −2.32145 | 1.32336 | LruC domain-containing |
| | | | | protein |
| Q8A826 | BT_1348 | −2.17801 | 2.67435 | CDP-abequose synthase |
| Q89ZP8 | BT_4328 | −2.167 | 3.63516 | Ribosomal RNA small |
| | | | | subunit methyltransferase E |
| Q89ZP9 | BT_4327 | −2.12513 | 5.58384 | DUF4836 family protein |
| Q8A149 | BT_3818 | −1.95031 | 1.38383 | Gliding motility lipoprotein |
| | | | | GldH |
| Q89YQ4 | BT_4677 | −1.90211 | 2.01555 | Beta-lactamase-inhibitor- |
| | | | | like PepSY-like domain- |
| | | | | containing protein |
| Q8A5G3 | BT_2276 | −1.88136 | 1.51383 | Al-2E family transporter |
| Q8A551 | BT_2391 | −1.74464 | 3.18011 | Hybrid Two-component |
| | | | | system |
| Q8A6C7 | BT_1957 | −1.70454 | 1.4706 | DUF4465 domain- |
| | | | | containing protein |
| Q8A8X3 | BT_1044 | −1.68106 | 3.56162 | Secreted |
| | | | | endoglycosidase, GH |
| | | | | family 18 |
| Q8A4A5 | BT_2698 | −1.65411 | 1.42781 | Unknown |
| Q8AAL2 | BT_0452 | −1.63773 | 1.32215 | SusC homolog |
| Q89YPO | BT_4691 | −1.59995 | 1.64528 | Ribosomal RNA large |
| | | | | subunit methyltransferase |
| | | | | F, rlmF |
| Q8A8X4 | BT_1043 | −1.59626 | 3.77015 | SusD homolog |
| Q8A6F8 | BT_1926 | −1.5911 | 1.87538 | OmpA/MotB domain |
| | | | | protein |
| Q8A5H2 | BT_2267 | −1.56716 | 1.5167 | Site-specific integrase |
| Q8A2R4 | BT_3241 | −1.50727 | 1.30956 | SusD homolog |

No function has been assigned to any of these three proteins; however, both NigD1 and NigD2 are annotated as NigD-like proteins. NigD-like proteins are a family of uncharacterized lipoproteins proteins found solely in Bacteroidota. It was determined that deletion of NigD1 (but not BT_1287 or NigD2) in the Δdma1 background restores wild-type levels of vesiculation, indicating that NigD1 is involved in controlling OMV production in Δdma1 (FIG. 4C). NigD1 is not predicted to be encoded within an operon; however, it is encoded nearby genes required for LPS biosynthesis and regulation (LpxB and FtsH) and phospholipid synthesis (CdsA). The transcriptomic and proteomic analyses did not identify these, or any other known genes involved in the biosynthesis of LPS or phospholipids, as differentially regulated between WT and Δdma1. However, the results suggest that NigD1 acts as a molecular switch for OMV biogenesis.

Dma1 Represents an Unprecedented Class of Anti-Sigma Factor that Spans Both the IM and OM.

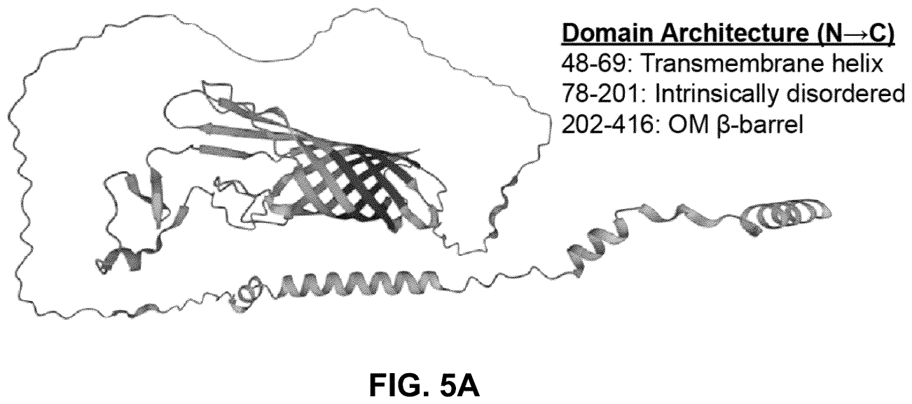

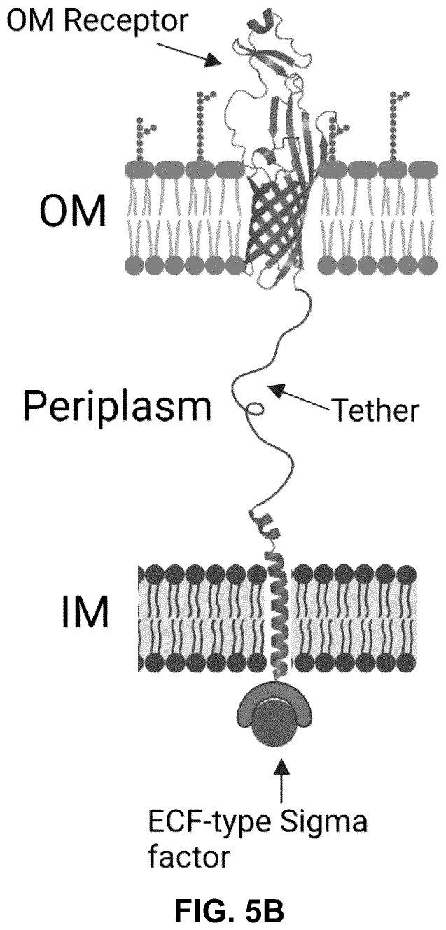

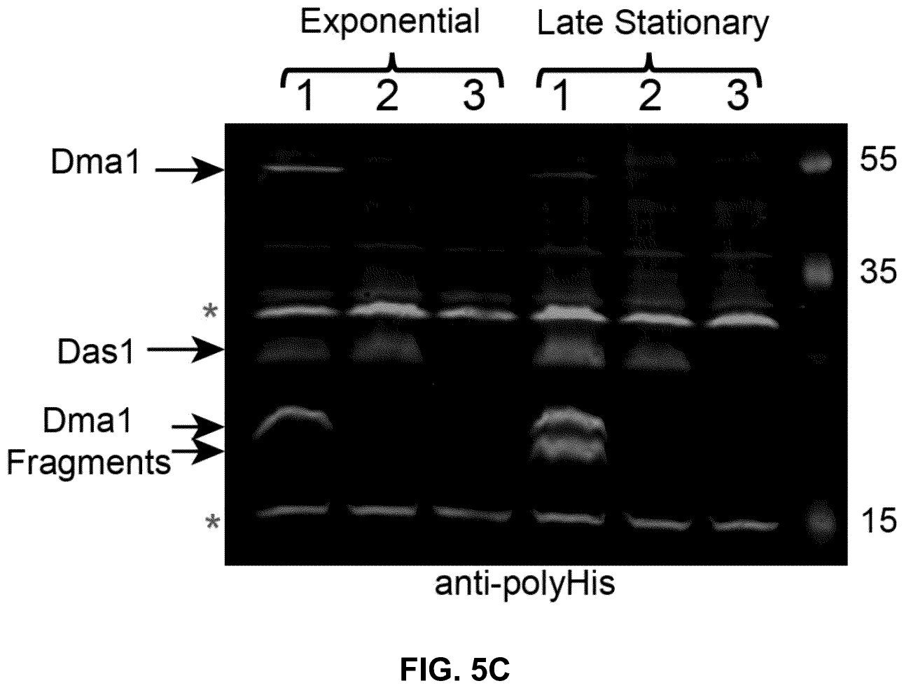

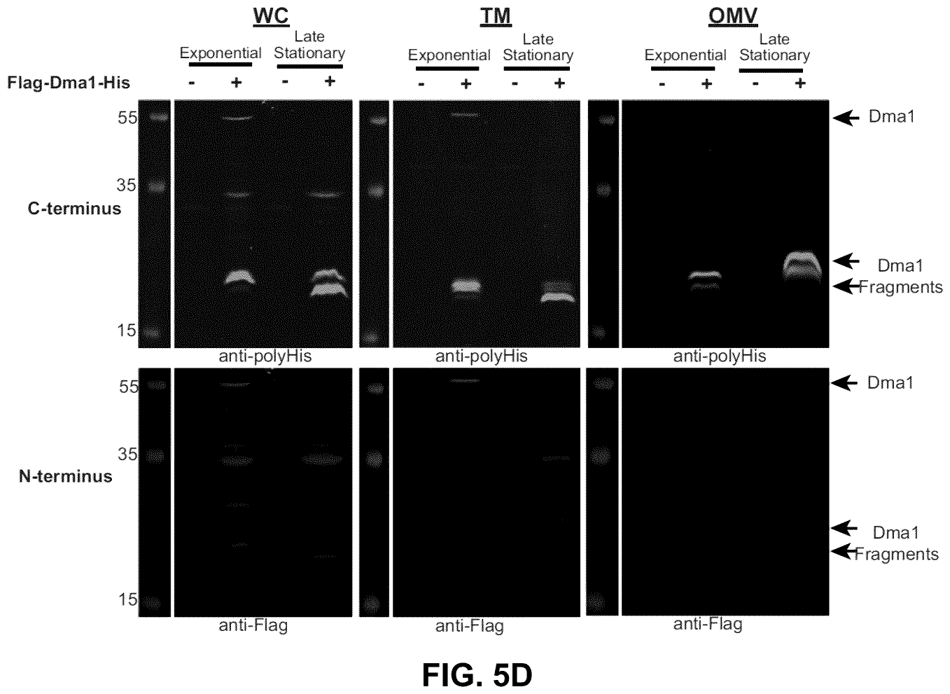

Canonical anti-sigma factors contain a cytoplasmic domain, responsible for the sequestration of their cognate sigma-factors at the IM, connected via a transmembrane region to a periplasmic sensor domain. Dma1 is annotated as an OMP_b-brl_2 domain-containing protein. Structural modeling with AlphaFold predicts that Dma1 contains an N-terminal alpha-helical transmembrane region and a C-terminal eight stranded β-barrel domain connected via a long intrinsically disordered region (FIG. 5A). Eight stranded β-barrel domain proteins are found in the OM of gram-negative bacteria. In Bf, Reo, was shown to directly sequester its cognate sigma factor via its N-terminal region in the cytoplasm, and it was shown that this is also the case for Dma1. Based on these observations, an unprecedented domain architecture for Dma1 is proposed, in which the C-terminal β-barrel is embedded in the OM; the N-terminal domain that interacts with the cognate sigma factor is in the cytoplasm anchored to the IM via an alpha-helical transmembrane helix; and both domains are tethered via the intrinsically disordered region that traverses the periplasm (FIG. 5B). Proteins with similar domain organizations have been suggested for Reo in B. fragilis, and MON98_03760 in P. vulgatus; however, the orientation of these proteins has not been experimentally addressed. To provide support for this model, western blots of Bt cells coexpressing C-terminally His-tagged Dma1 and Das1 were performed. Cells were harvested during the exponential and late stationary phase. Expression of Dma1 was enhanced when expressed alongside Das1. Wild-type Bt and cells expressing only Das1 were employed as controls. During exponential phase growth, a band corresponding to full-length (˜55 kDa) and a C-terminal-containing fragment (˜20 kDa) of Dma1 were detected (FIG. 5C). At stationary phase, full-length Dma1 was no longer detected, with the concomitant appearance of an additional smaller fragment (˜17 kDa). These results suggest that Dma1 is temporally regulated (FIG. 5C). Next, cells expressing Dma1 tagged with both a C-terminal 10×His tag and an N-terminal 3×FLAG tag were generated. Whole cells (WCs), TMs, and OMVs were analyzed by western blot. Full-length Dma1 (˜55 kDa) was detected in the WC and TM fractions with both antibodies, confirming that Dma1 is translated and translocated as a complete and single polypeptide (FIG. 5D). Only C-terminal fragments were detected in the OMVs using the anti-His antibodies, indicating that, as predicted, the β-barrel domain is localized in the OM (FIG. 5D). In addition, the conclusion is supported by the MS analysis of wild-type OMVs, which only detected peptides containing the predicted C-terminal β-barrel of Dma1 (Table 4). Taken together, the results provide strong evidence that Dma1 is an anti-sigma factor that spans both membranes, directly connecting the exterior of the cell with its cytoplasm. Based on this, it is proposed that Dma1 is the founding member of the Dual Membrane-spanning Anti-sigma factor (Dma) family.

TABLE 4
List of tryptic peptides of Dma1 identified in the OMV fraction.

| Start | End | | Posterior Error |
|---|---|---|---|
| Position | Position | Sequence | Probability |
| 153 SEQ ID | NO: 55 164 | EKEEVEPVEETK | 1.62E−05 |
| 187 SEQ ID | NO: 56 195 | DKLHIPAEK | 0.0033255 |
| 189 SEQ ID | NO: 57 195 | LHIPAEK | 0.038234 |
| 259 SEQ ID | NO: 58 279 | QANQVVDMEHHQPISFGLSVR | 3.29E−14 |
| 285 SEQ ID | NO: 59 302 | GFSVETGLTYTLLSSDAK | 1.01E−36 |
| 314 SEQ ID | NO: 60 322 | LHYLGIPLK | 6.66E−06 |
| 323 SEQ ID | NO: 61 330 | ANWNFLDK | 0.0044146 |
| 323 SEQ ID | NO: 62 331 | ANWNFLDKK | 2.04E−05 |
| 356 SEQ ID | NO: 63 377 | ETVKPLQFSVSGAVGAQFNATK | 1.58E−09 |
| 378 SEQ ID | NO: 64 401 | RVGIYVEPGVAYFFDDGSDVQTIR | 4.09E−08 |
| 379 SEQ ID | NO: 65 401 | VGIYVEPGVAYFFDDGSDVQTIR | 5.89E−08 |
| 402 SEQ ID | NO: 66 415 | KENPFNFNIQAGIR | 6.49E−12 |
| 403 SEQ ID | NO: 67 415 | ENPFNFNIQAGIR | 2.13E−09 |

### Dual Membrane-Spanning Anti-Sigma Factors are Present Throughout Bacteroidota.

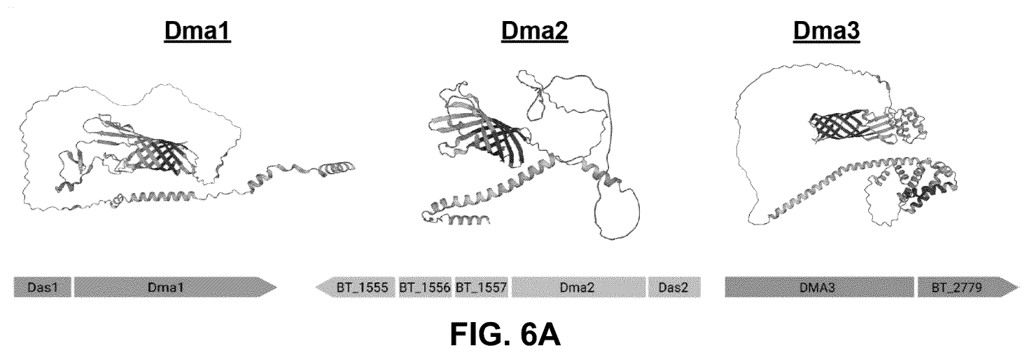

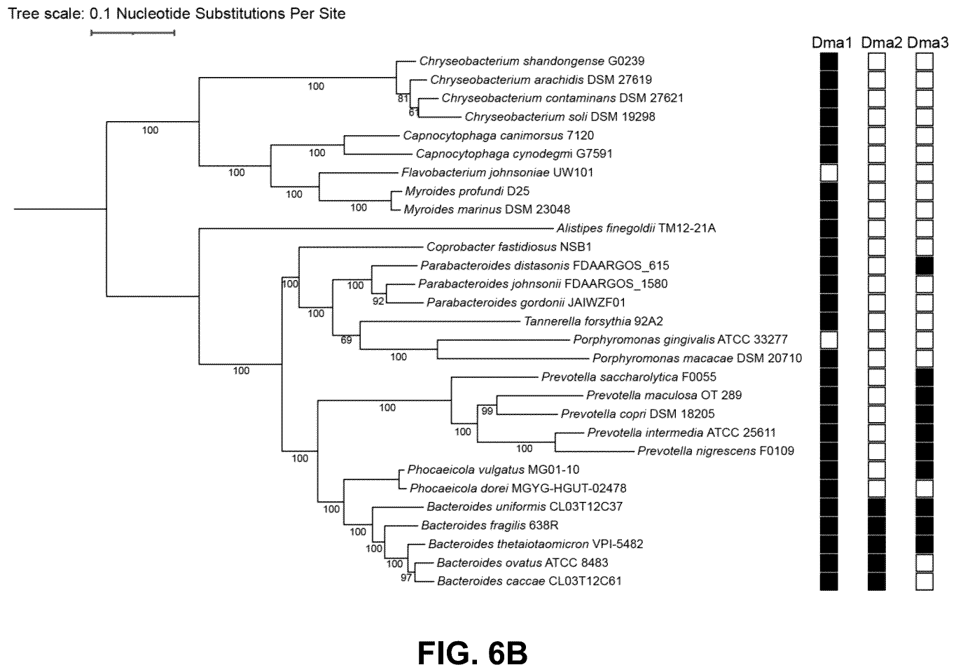

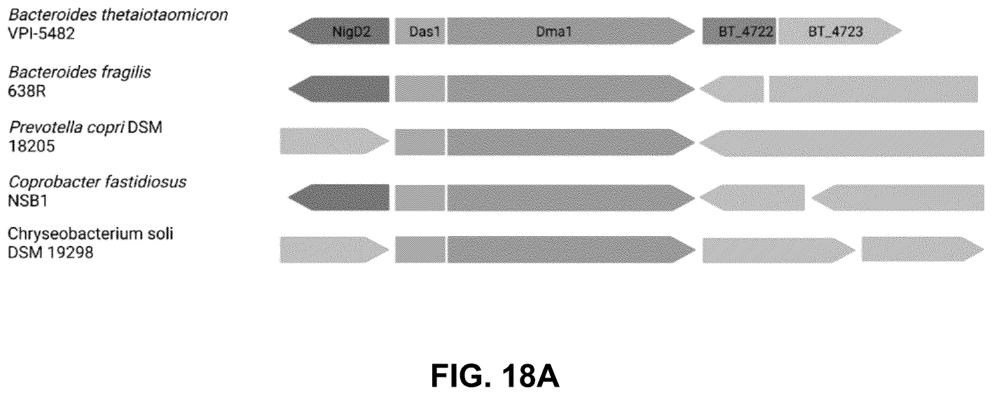

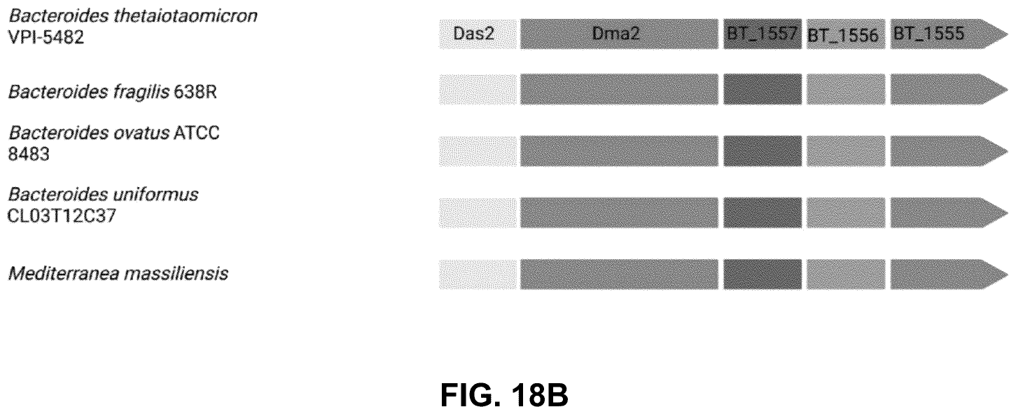

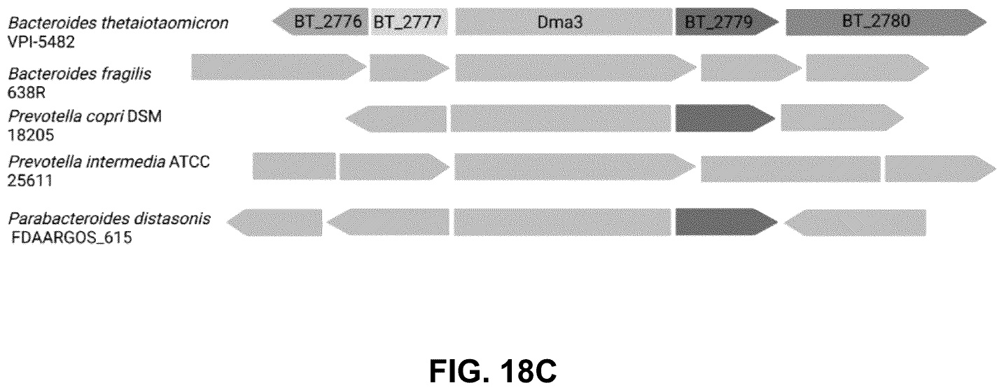

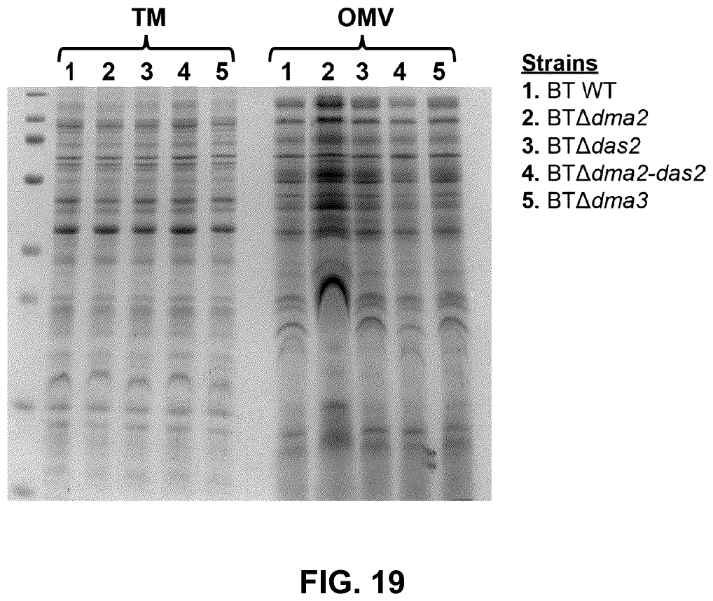

To determine whether proteins with similar domain architecture exist in Bt, sigma/anti-sigma pairs containing anti-sigma factors encoding a predicted β-barrel domain were searched for. Two additional proteins were detected and identified in Bt, Dma2 (BT_1558) and Dma3 (BT_2778), that are structurally similar to Dma1 (FIG. 6A). Dma2 is encoded adjacent to an ECF21 family sigma factor, BT_1559 (Das2), while Dma3 is more complex, as it is predicted to contain the sigma factor fused to the rest of the polypeptide at the N terminus (FIG. 6A). A bioinformatic analyses showed that Dma1 is present in almost all Bacteroidota, while Dma2 and Dma3 are less prevalent. FIG. 6B shows a phylogenetic analysis containing select members of the Bacteroidota representing various classes ranging from mammalian gut commensals to soil-dwelling microbes. Dma1 is present in almost all Bacteroidota chosen (27/29), while Dma2 (5/29) and Dma3 (10/29) were less prevalent (FIG. 6B). Interestingly, Dma2 is only present in members of the genus Bacteroides, suggesting that this protein was recently acquired in the genus. Dma3 on the other hand is predominantly found in Bacteroides and Prevotella (FIGS. 6B and 18). No structurally similar proteins were identified in gram-negative bacteria outside of Bacteroidota. To determine whether Dma2 and Dma3 may also modulate OMV biogenesis in Bt, clean deletion mutants were generated in each gene and their protein profiles were analyzed by SDS-PAGE. Mutation of dma2 produced a phenotype similar to that of Δdma1, which suggests that Dma2 also potentially modulates OMV biogenesis. Deletion of its cognate sigma factor das2 also restores the WT electrophoretic profile (FIG. 19). Mutation of dma3 revealed no detectable phenotype by SDS-PAGE (FIG. 19). Additional studies are needed to validate the potential impacts of Dma2 and Dma3 on OMV biogenesis; however, these are outside the scope of this manuscript.

### DISCUSSION

Since their discovery in the 1960s, many important roles have been proposed for OMVs. However, to date, very little is known about the mechanism(s) of bacterial vesiculation and its regulation. In Example 1, it was aimed to advance the understanding of the mechanism of OMV biogenesis in Bacteroidota. To this aim, a screening methodology was developed to identify genes involved in OMV biogenesis. The Dma family was identified, which is shown to be a class of anti-sigma factors with an unprecedented domain organization. These proteins span both membranes, possessing an extracellular and a cytoplasmic domain connected via a large, intrinsically disordered region that crosses the periplasm. It is shown that inactivation of Dma1 (BT_4721) or Dma2 (BT_1558) results in hypervesiculation in Bt.

It was previously shown that labeling OM and OMV-specific proteins with distinct fluorescent markers is an effective way to visualize OMVs and distinguish them from by-products of cell lysis. In Example 1, an OMV marker was labeled with NLuc instead of fluorescent proteins, which allowed quantification of OMV production in vitro in a high throughput format (FIG. 1C). Screens attempting to identify genes involved in OMV biogenesis have been performed, however, these were unable to establish causal associations between specific genes and OMV formation. It is speculated that this is due to their use of nonspecific markers, like LPS, OmpA, or phospholipids, as a readout for OMV production. Many of the genes identified in these studies led to membrane destabilization, which confounded the interpretation of the results, due to their inability to differentiate genuine OMVs from cell lysis. Although OMV cargo selection is not common in all bacteria, the methods developed here can be adapted to conduct OMV screens in other bacterial species.

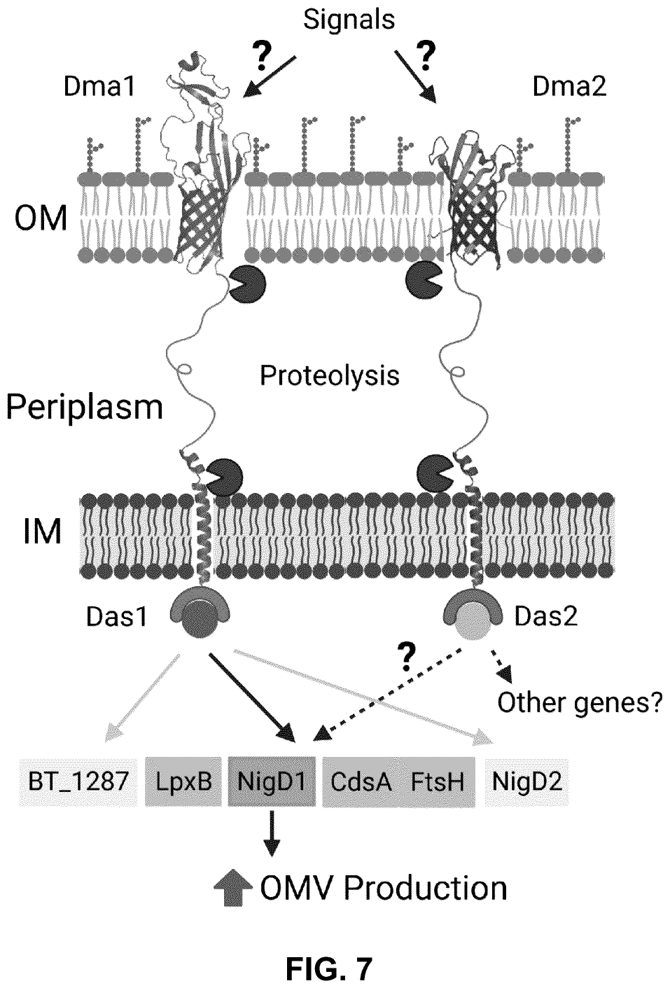

The model for regulation of OMV biogenesis in Bt is summarized in FIG. 7. It is proposed that members of the Dma family sense extracellular stimuli or perturbations in the OM via their C-terminal β-barrel domain, which triggers a series of proteolytic events that liberates their cognate sigma factors to modulate gene expression and induce OMV production (FIG. 7). By employing transcriptomic and proteomic analyses, it is demonstrated that NigD1 (BT_4005) is required for the induction of vesiculation in the absence of Dma1. NigD-like proteins are ubiquitous among Bacteroidota, but their functions have not been defined. NigD1 is encoded adjacent to genes required for biosynthesis and regulation of LPS and phospholipids. The transcriptomic and proteomic analyses indicate that these genes/proteins are not differentially regulated between the wild type and Δdma1. This suggests the presence of currently unknown mechanisms where NigD1 controls the amount of LPS, proteins, and lipids allocated to OMVs. However, Bacteroidota membranes are rich in sphingolipids and other lipids not commonly found in bacteria. More knowledge about the regulation of LPS and lipid biosynthesis will be required to fully understand how Dma1, Dma2 and NigD1 control OMV biogenesis in Bacteroides. NigD2 (BT_4719) and BT_1287 are part of the Dma1 regulon, but they are dispensable for the hyper-vesiculation phenotype. This strongly suggests that Dma1 regulates other processes in Bt.

Previous studies have investigated orthologs of Dma1. In B. fragilis, deletion of Reo, Dma1 ortholog, was shown to increase fitness in response to oxidative stressors. However, herein it was found that Dma1 does not seem to play the same role in Bt (FIG. 17). In Phocaeicola vulgatus, mutation of the Dma1 ortholog, M098_03760, conferred increased protection against the antimicrobial toxin BcpT. This occurs by increasing LPS O-antigen length and preventing BcpT from binding lipid A core and destabilizing the OM. The structure of LPS was not analyzed in this work, but reports suggest that Bt lacks LPS, and instead makes lipooligosaccharide (LOS). Therefore, Dma1 is not likely to induce a similar change. In Bt, the Dma family impacts OMV biogenesis; however, members of the Dma family likely play diverse roles in different species within Bacteroidota. Since OMVs have been implicated in stress response, it would be interesting to determine whether the fitness advantages observed in other studies are somehow related to increased vesiculation.

Dma1 is the first protein shown to span both membranes of a gram-negative bacteria (FIG. 5). As anti-sigma factors, the Dma family represents a unique class of regulatory protein. The canonical cell surface signaling (CSS) system, like the Fec system in E. coli, consists of a TonB-dependent OM receptor (FecA), which senses external stimuli, an anti-sigma factor (FecR), to relay the signal, and a sigma factor (Fecl), to modulate gene expression. Members of the Dma family are distinct from this model because they encode the OM receptor and anti-sigma factor in a single polypeptide (FIG. 5). To date, such structures have yet to be reported. Bacteroides spp. distinctively encode an expanded repertoire of transcriptional regulators, specifically hybrid two-component systems and ECF-type sigma factors, many of which are uncharacterized. The identification of the Dma family in Bacteroidota suggests that these organisms have evolved additional modes of transcriptional regulation, many of which are yet to be described.

The unprecedented domain organization of the Dma family raises fundamental questions regarding their translocation and assembly. In gram-negative bacteria, β-barrel Outer Membrane Proteins (OMPs) are trafficked to the OM by translocation through the Sec system, where periplasmic chaperones, like surA, shuttle the unfolded OMPs to the β-barrel Assembly Machine (BAM) for insertion into the OM. Since Dma1 has domains inserted into both membranes, it is unclear whether Dma1 is localized via the BAM complex. Bacteroidota may have evolved additional systems specifically to ensure the proper localization of members of the Dma family, and requires validation.

The presence of Dma1 proteolytic fragment provides clues about Dma1 regulation. In E. coli and similar organisms, there are typically two proteolytic events that occur at the inner and outer leaflet of the IM to inactivate anti-sigma factors. Two IM proteases, DegS and RseP, involved in RIP have been characterized. Potential orthologs of these IM proteases are predicted to be present in Bt. Understanding the signals that trigger Dma1 proteolysis and the enzymes involved in the process is also contemplated.

The screening led to the identification of the Dma family. However, multiple mutants displaying hyper- and hypovesiculation phenotypes were also identified. The investigation of these mutants will further understanding of the bacterial vesiculation mechanisms and will lead to identification of the elusive machinery responsible for OMV biogenesis.

### Materials and Methods

### Bacterial Strains and Growth Conditions.

Strains, oligonucleotides, and plasmids are described in Table 5. E. coli was grown aerobically at 37° C. in Luria-Bertani (LB) medium. Bacteroides strains were grown in an anaerobic chamber (Coy Laboratories) at 37° C. containing an atmosphere of 10% H2, 5% CO2, 85% N₂. Bacteroides were cultured in brain heart infusion (BHI) medium (Fisher Scientific) supplemented with 5 μg/mL hemin and 1 μg/mL vitamin K3. When applicable, antibiotics were used as follows: 100 μg/mL ampicillin, 200 μg/mL gentamicin, 25 μg/mL erythromycin, and 10 μg/mL tetracycline.

TABLE 5
List of strains, plasmids, and oligonucleotides used and identified in the
present disclosure.
Strains used in this study

| Name | Strain | Features | Reference/Source |
|---|---|---|---|
| Escherichia coli | s17-1λpir | Conjugation donor strain to | Jeffrey I. Gordon |
| | | introduce plasmids into B. | Laboratory |
| | | thetaiotaomicron | |
| Escherichia coli | BL21 | Expression and isolation of | Tracey Raivio |
| | | heterologous proteins | Laboratory |
| Escherichia coli | BL21 pGSTag | Expresses GST under IPTG | This study |
| | | inducible promoter | |
| Escherichia coli | BL21 pGSTag_Dma1(1:40) | Expresses the N-terminal 40 | This study |
| | | aa of Dma1 fused to the C- | |
| | | terminus of GST | |
| Escherichia coli | BL21 pET32a-TRXtag | Expresses TRX under IPTG | This study |
| | | inducible promoter | |
| Escherichia coli | BL21 pET32a-TRXtag_ | Expresses Das1 fused to the | This study |
| | Das1 | C-terminus of TRX | |
| B. thetalotaomicron | VPI-5482 | Wild-type strain. Erm^{S} | Jeffrey I. Gordon |
| | | | Laboratory |
| B. thetalotaomicron | VPI-5482 pwwBolNL-Nluc | Expressing B. ovatus | This study |
| | | inulinase fused to Nluc | |
| B. thetalotaomicron | VPI-5482 Δdas1 | das1 (bt_4720) deletion | This study |
| | | mutant | |
| B. thetaiotaomicron | VPI-5482 Δdma1 | dma1 (bt_4721) deletion | This study |
| | | mutant | |
| B. thetaiotaomicron | VPI-5482 Δdas1-dma1 | das1 (bt_4720)-dma1 | This study |
| | | (bt_4721) double deletion | |
| | | mutant | |
| B. thetaiotaomicron | VPI-5482 Δdas2 | das2 (bt_1559) deletion | This study |
| | | mutant | |
| B. thetalotaomicron | VPI-5482 Δdma2 | dma2 (bt_1558) deletion | This study |
| | | mutant | |
| B. thetalotaomicron | VPI-5482 Δdas2-dma2 | das2 (bt_1559)-dma2 | This study |
| | | (bt_1558) double deletion | |
| | | mutant | |
| B. thetalotaomicron | VPI-5482 Δdas1 | das1 (bt_4720) deletion | This study |
| | | mutant, complemented | |
| B. thetalotaomicron | VPI-5482 Δdma1 | dma1 (bt_4721) deletion | This study |
| | pwwDma1-His | mutant, complemented | |
| B. thetaiotaomicron | VPI-5482 Δdas1-dma1 | das1 (bt_4720)-dma1 | This study |
| | pwwDas1-Dma1 | (bt_4721) double deletion | |
| | | mutant, complemented | |
| B. thetaiotaomicron | VPI-5482 Δdma1 | dma1 (bt_4721) deletion | This study |
| | pwwFLAG-Dma1-His | mutant expressing | |
| | | pwwFLAG-Dma1-His | |

Plasmids used in this study

| Name | Resistance | Features | Reference/Source |
|---|---|---|---|
| pSAM-Bt | Amp^{R}, Erm^{R} | Vector base to perform | Goodman et al., |
| | | transposon mutagenesis | 2009 |
| pSAM-Bt_Tet | Amp^{R}, Tet^{R} | pSAM-Bt backbone | This work |
| | | containing tetracycline | |
| | | resistance cassette | |
| pSIE1 | Amp^{R}, Erm^{R}, aTc^{S} | P1T_DP-GH023_ss-bte1; | Bencivenga-Barry |
| | | P1T_DP-GH023_ss-bfe1; | et al., 2020 |
| | | P_{BFP1E6}_tetR | |
| pSIE1_das1 | Amp^{R}, Erm^{R}, aTc^{S} | pSIE1 deletion construct for | This study |
| | | B. thetaiotaomicron VPI- | |
| | | 5482 das1 | |
| pSIE1_dma1 | Amp^{R}, Erm^{R}, aTc^{S} | pSIE1 deletion construct for | This study |
| | | B. thetaiotaomicron VPI- | |
| | | 5482 dma1 | |
| pSIE1_das1-dma1 | Amp^{R}, Erm^{R}, aTc^{S} | pSIE1 deletion construct for | This study |
| | | B. thetaiotaomicron VPI- | |
| | | 5482 das1& dma1 | |
| pSIE1_das2 | Amp^{R}, Erm^{R}, aTc^{S} | pSIE1 deletion construct for | This study |
| | | B. thetaiotaomicron VPI- | |
| | | 5482 das2 | |
| pSIE1_dma2 | Amp^{R}, Erm^{R}, aTc^{S} | pSIE1 deletion construct for | This study |
| | | B. thetaiotaomicron VPI- | |
| | | 5482 dma2 | |
| pSIE1_das2-dma2 | Amp^{R}, Erm^{R}, aTc^{S} | pSIE1 deletion construct for | This study |
| | | B. thetaiotaomicron VPI- | |
| | | 5482 das2 & dma2 | |
| pSIE1_BT1287 | Amp^{R}, Erm^{R}, aTc^{S} | pSIE1 deletion construct for | This study |
| | | B. thetaiotaomicron VPI- | |
| | | 5482 BT_1287 | |
| pSIE1_nigD1 | Amp^{R}, Erm^{R}, aTc^{S} | pSIE1 deletion construct for | This study |
| | | B. thetaiotaomicron VPI- | |
| | | 5482 NigD1 (BT_4005) | |
| pSIE1_nigD2 | Amp^{R}, Erm^{R}, aTc^{S} | pSIE1 deletion construct for | This study |
| | | B. thetaiotaomicron VPI- | |
| | | 5482 NigD2 (BT_4719) | |
| pWW3452 | Amp^{R}, Erm^{R} | AmpR-ermG-RP4/R6K- | Whitaker et al., |
| | | [P_BfP1E6-RBSIp-LPGFP- | 2017 |
| | | tag-Term]-NBU2, AmpR | |
| | | ErmR | |
| pWW3867 | Amp^{R}, Erm^{R} | AmpR-ermG-RP4/R6K- | Whitaker et al., |
| | | [P_BT1311-RBSphageGFP- | 2017 |
| | | tag-Term]-NBU2, AmpR | |
| | | ErmR | |
| pwwBolNL-Nluc | Amp^{R}, Erm^{R} | pWW3867containing | This study |
| | | Inulinase-Nluc fusion | |
| pwwNLuc | Amp^{R}, Erm^{R} | pWW3867containing Nluc | This study |
| pwwEV | Amp^{R}, Erm^{R} | pWW3867 backbone | Whitaker et al.., |
| | | | 2017 |
| pwwDas1-His | Amp^{R}, Erm^{R} | pWW3867 containing Das1 | This study |
| | | with C-terminal 6xHis tag | |
| pwwDma1-His | Amp^{R}, Erm^{R} | pWW3867 containing Dma1 | This study |
| | | with C-terminal 10xHis tag | |
| pwwFLAG-Dma1-His | Amp^{R}, Erm^{R} | pWW3867 containing Dma1 | This study |
| | | with an N-terminal 3xFlag | |
| | | tag and a C-terminal 10xHis | |
| | | tag | |
| pwwDas1-Dma1-His | Amp^{R}, Erm^{R} | pWW3867 containing Das1 | This study |
| | | and Dma1 with C-terminal | |
| | | His tags | |
| pGSTag | Amp^{R} | Expresses GST and is used | Ron et al. 1992 |
| | | to create GST fusion | |
| | | proteins | |
| pET32a-TRXtag | Amp^{R} | Expresses TRX and is used | Bennett et al. |
| | | to create TRX fusion | 2002 |
| | | proteins | |

Oligonucleotides used in this study

| Name | Sequence | Template | Description |
|---|---|---|---|
| F_Nluc | GTCTTCACACTCGAAGATTTC | pLenti6.2-Nanoluc-ccdB | For cloning into |
| SEQ ID NO: 1 | | vector | pWW3867 |
| R_RpoD_Nluc(6His) | TCGAGCTAATCAGCTAGGATTT | pLenti8.2-Nanoluc-ccdB | For cloning into |
| SEQ ID NO: 2 | AGTGATGATGATGATGATGACC | vector | pWW3867 |
| | CGCCAGAATGCGTTC | | |
| F_BolN-RpoD | TCCAAATGTGTTTTTAAAGAAT | B. ovatus ATCC 8483 | For cloning into |
| SEQ ID NO: 3 | GAAGATAAATAAATTCTTAATA | genomic DNA | pWW3867 |
| | AGCGG | | |
| R_BolN(SF)Nluc | AAATCTTCGAGTGTGAAGACAG | B. ovatus ATCC 8483 | For cloning into |
| SEQ ID NO: 4 | ATCCTCCTCCTCCTTTCTTAGC | genomic ONA | pWW3867 |
| | GCTTAGATAATG | | |
| F_pww-NLuc-His | GTCTTCACACTCGAAGATTTC | pLenti6.2-Nanoluc-ccdB | For cloning into |
| linear | | vector | pWW3867 |
| SEQ ID NO: 5 | | | |
| R_pww-NLuc | TCGAGCTAATCAGCTAGGATTT | plenti6.2-Nanoluc-ccd8 | For cloning into |
| SEQ ID NO: 6 | AGTGATGATGATGATGATGACC | vector | pWW3867 |
| | CGCCAGAATGCG | | |
| F_BT4720-Downstream | GCGGCCGCTCTAGAACTAGTCGC | B. thetaiotaomicron VPI- | For cloning into |
| SEQ ID NO: 7 | CTTCGTCGTTATG | 5482 genomic DNA | pSIE1 |
| R_BT4720-Downstream | TTTCTCTTCCATATCTCTACTT | B. thetaiotaomicron VPI- | For cloning into |
| SEQ ID NO: 8 | GCTTATACACGTGTTTACC | 5482 genomic DNA | pSIE1 |
| F_BT4720-Upstream | GTAAACACGTGTATAAGCAAGT | B. thetaiotaomicron VPI- | For cloning into |
| SEQ ID NO: 9 | AGAGATATGGAAGAGAAAGAAT | 5482 genomic ONA | pSIE1 |
| | TATG | | |
| R_BT4720-Upstream | GATTAGCATTATGAGGATCCAT | B. thetaiotaomicron VPI- | For cloning into |
| SEQ ID NO: 10 | TGCTGATCATTGGGTATG | 5482 genomic DNA | pSIE1 |
| F_BT4721_Up | GCGGCCGCTCTAGAACTAGTTG | B. thetaiotaomicron VPI- | For cloning into |
| SEQ ID NO: 11 | GTCAGGATCTCTTTTATTTCTC | 5482 genomic DNA | pSIE1 |
| | TTACCT | | |
| R_BT4721_Up | ATCTCTACCTATCCGTTATTCA | B. thetaiotaomicron VPI- | For cloning into |
| SEQ ID NO: 12 | TTATCCATTC | 5482 genomic DNA | pSIE1 |
| F_BT4721_Down | AATAACGGATAGGTAGAGATTA | B. thetaiotaomicron VPI- | For cloning into |
| SEQ ID NO: 13 | GAACCCGGTGTTGCTTATTTCT | 5482 genomic DNA | pSIE1 |
| | TTGATGATG | | |
| R_BT4721_Down | AAGATTAGCATTATGAGGATCC | B. thetaiotaomicron VPI- | For cloning into |
| SEQ ID NO: 14 | GGTGACGTATGTTTCAGTGTTC | 5482 genomic DNA | pSIE1 |
| R_BT4720/21- | ATATAGCTCAACTATATTGATT | B. thetaiotaomicron VPI- | For cloning into |
| Downstream | GCTTATACACGTGTTTACC | 5482 genomic DNA | pSIE1 |
| | | | SEQ ID NO: 15 |
| F_BT4721/20-Upstream | GTAAACACGTGTATAAGCAATC | B. thetaiotaomicron VPI- | For cloning into |
| SEQ ID NO: 16 | AATATAGTTGAGCTATATATTT | 5482 genomic DNA | pSIE1 |
| | TAAAAGC | | |
| F_BT1558 Downsteam | GCGGCCGCTCTAGAACTAGTCA | B. thetaiotaomicron VPI- | For cloning into |
| SEQ ID NO: 17 | GGCACAGTGTCATAG | 5482 genomic DNA | pSIE1 |
| R_BT1558 Downstream | CGCTGATGCCGGGATGACTGTC | B. thetaiotaomicron VPI- | For cloning into |
| SEQ ID NO: 18 | CTTATAAAAATGAAAAAAACAC | 5482 genomic DNA | pSIE1 |
| | TTATG | | |
| F_BT1558 Upstream | TTTTTTCATTTTTATAAGGACA | B. thetaiotaomicron VPI- | For cloning into |
| SEQ ID NO: 19 | GTCATCCCGGC | 5482 genomic DNA | pSIE1 |
| R_ST1558 Upstream | GATTAGCATTATGAGGATCCCG | B. thetaiotaomicron VPI- | For cloning into |
| SEQ ID NO: 20 | TGCTGCAGCTG | 5482 genomic DNA | pSIE1 |
| F_BT1559 Downstream | GGGGCCGCTCTAGAACTAGTGC | B. thetaiotaomicron VPI- | For cloning into |
| SEQ ID NO: 21 | TTTCAGGGGGATG | 5482 genomic DNA | pSIE1 |
| R_BT1559 Downstream | ACACCCTCTATGCCAAACAATG | B. thetaiotaomicron VPI- | For cloning into |
| SEQ ID NO: 22 | AAATAACCGATCTGTTTCG | 5482 genomic DNA | pSIE1 |
| F_BT1558 Upstream | GAAACAGATCGGTTATTTCATT | B. thetaiotaomicron VPI- | For cloning into |
| SEQ ID NO: 23 | GTTTGGCATAGAGGG | 5482 genomic DNA | pSIE1 |
| R_BT1859 Upstream | GATTAGCATTATGAGGATCCTT | B. thetaiotaomicron VPI- | For cloning into |
| SEQ ID NO: 24 | AGGTACCCGCAAG | 5482 genomic DNA | pSIE1 |
| F_BT1287_Dwa | GCGGCCGCTCTAGAACTAGTCC | B. thetaiotaomicron VPI- | For cloning into |
| SEQ ID NO: 25 | GGTCATCCTTTACG | 5482 genomic DNA | pSIE1 |
| R_BT1287_Dwn | TAAATAATTAAACAGTCACCTT | B. thetaiotaomicron VPI- | For cloning into |
| SEQ ID NO: 26 | TAGTTAAAGTTTTCATACTTGT | 5482 genomic DNA | pSIE1 |
| | TAAAC | | |
| F_ST1287_Up | GTATGAAAACTTTAACTAAAGG | B. thetaiotaomicron VPI- | For cloning into |
| SEQ ID NO: 27 | TGACTGTTTAATTATTTACAG | 5482 genomic DNA | pSIE1 |
| R_ST1287_Up | GATTAGCATTATGAGGATCCAG | B. thetaiotaomicron VPI- | For cloning into |
| SEQ ID NO: 28 | AGGCGAATGCG | 5482 genomic DNA | pSIE1 |
| F_BT4005_Dwn | GCGGCCGCTCTAGAACTAGTTA | B. thetaiotaomicron VPI- | For cloning into |
| SEQ ID NO: 29 | GGAAATCCTACGGTAG | 5482 genomic DNA | pSIE1 |
| R_BT4005_Dwn | AGTTATTTATAAAAGTTTTGAT | B. thetaiotaomicron VPI- | For cloning into |
| SEQ ID NO: 30 | TTGTTCCTCTCTTTTTAATTC | 5482 genomic DNA | pSIE1 |
| F_BT4005_Up | TTAAAAAGAGAGGAACAAATCA | B. thetaiotaomicron VPI- | For cloning into |
| SEQ ID NO: 31 | AAACTTTTATAAATAACTAAAC | 5482 genomic DNA | pSIE1 |
| | CTAAAC | | |
| R_BT4005_Up | GATTAGCATTATGAGGATCCAC | B. thetaiotaomicron VPI- | For cloning into |
| SEQ ID NO: 32 | CTGTTCCTCGC | 5482 genomic DNA | pSIE1 |
| F_BT4719_DWR | GCGGCCGCTCTAGAACTAGTGA | B. thetaiotaomicron VPI- | For cloning into |
| SEQ ID NO: 33 | GATGCTGTTTTTGCC | 5482 genomic DNA | pSIE1 |
| R_BT4719_Dwn | AAAAAACATTTTTATAGTATAT | B. thetaiotaomicron VPI- | For cloning into |
| SEQ ID NO: 34 | CTAGTAATTATAGAAACACTC | 5482 genomic DNA | pSIE1 |
| F_BT4719_Up | TGTTTCTATAATTACTAGATAT | B. thetaiotaomicron VPI- | For cloning into |
| SEQ ID NO: 35 | ACTATAAAAATGTTTTTTAATG | 5482 genomnic DNA | pSIE1 |
| | AGTACTATTTAC | | |
| R_ST4719_Up | GATTAGCATTATGAGGATCCTC | B. thetaiotaomicron VPI- | For cloning into |
| SEQ ID NO: 36 | CTTTTCTTCCATATCTCTAC | 5482 genomic DNA | pSIE1 |
| F_RDD_ST4720-His | TCCAAATCTGTTTTTAAAGAAT | B. thetaiotaomicron VPI- | For cloning inio |
| SEQ ID NO: 37 | GGAAGAATTTGAGTTGTCG | 5482 genomic DNA | pWW3867 |
| R_RpoD_BT4720-His | TCGAGCTAATCAGCTAGGATCT | B. thetaiotaomicron VPI- | For cloning into |
| SEQ ID NO: 38 | AGTGATGATGATGATGATGACC | 5482 genomic DNA | pWW3867 |
| | TCCGTTATTCATTATCCATTCT | | |
| | TTTAC | | |
| F_pwwRpoD_BT4721- | CCAAATCTGTTTTTAAAGAATG | B. thetaiotaomicron VPI- | For cloning into |
| His SEQ ID NO: 39 | GAAGAGAAAGAATTATGG | 5482 genomic DNA | pWW3867 |
| R_pwwRpoD_BT4721- | TCGAGCTAATCAGCTAGGATCT | B. thetaiotaomicron VPI- | For cloning into |
| His | AATGATGATGATGGTGATGATG | 5482 genomic DNA | pWW3867 |
| SEQ ID NO: 40 | ATGATGATGACCATAAGTAAGT | | |
| | CGGATACC | | |
| R_RpOD_BT4720/21- | TTTCTCTTCCATATCTCTACCT | B. thetaiotaomicron VPI- | For cloning into |
| SEQ ID NO: 41 | AGTGATGATGATGATGATGACC | 5482 genomnic DNA | pWW3867 |
| | TCCGTTATTCATTATCCATTCT | | |
| | TTTAC | | |
| F_pwwwRpoD_BT4721/ | CATCATCACTAGGTAGAGATAT | B. thetaiotaomicron VPI- | For cloning into |
| 20-His SEQ ID | GGAAGAGAAAGAATTATGG | 5482 genomic DNA | pWW3867 |
| NO: 42 | | | |
| F_FLAG-4721_lin | ATGGATTACAAGGACGATG | pWW3452 DNA | For cloning into |
| SEQ ID NO: 43 | | | pWW3452 |
| R_N-term_3x- | ATCCATAATTCTTTCTCTTCTC | pWW3452 DNA | For cloning into |
| FLAG_BT4721 | CAGATTTATCATCATCG | SEQ ID NO: 44 | pWW3452 |
| F_Phg_FLAG-BT4721- | ACGATGATGATAAATCTGGAGA | B. thetaiotaomicron VPI- | For cloning into |
| His SEQ ID NO: 45 | AGAGAAAGAATTATGGATG | 5482 genomic DNA | pWW3452 |
| R_pwwRpoD_BT4721- | TCGAGCTAATCAGCTAGGATCT | B. thetaiotaomicron VPI- | For cloning into |
| His | AATGATGATGATGGTGATGATG | 5482 genomnic DNA | pWW3452 |
| SEQ ID NO: 46 | ATGATGATGACCATAAGTAAGT | | |
| | CGGATACC | | |
| F_pGSTag_lin | GCAGAACGTCGCGAA | pGSTag DNA | For cloning into |
| | SEQ ID NO: 47 | | pGSTag |
| R_pGSTag_lin | AGATCCACGCGGAAC | pGSTag DNA | For cloning into |
| | SEQ ID NO: 48 | | pGSTag |
| F_GST-BT4721_1-80aa | ATCTGGTTCCGCGTGGATCTAT | B. thetaiotaomicron VPI- | For cloning into |
| SEQ ID NO: 49 | GGAAGAGAAAGAATTATGG | 5482 genomic DNA | pGSTag |
| R_GST-BT4721 1-40aa | AGAATTTCGCGACGTTCTGCCT | B. thetaiotaomicron VPI- | For cloning into |
| SEQ ID NO: 50 | ACCTCTCCACAGG | 5482 genomic DNA | pGSTag |
| F_pET32a-TRX_lin | CATCATCATCATCATCATTGAG | pET32a-TRXtag | For cloning into |
| SEQ ID NO: 51 | | | pET32a-TRXtag |
| R_pET32a-TRX_lin | CTTGTCGTCGTCGTC | pET32a-TRXtag | For cloning into |
| SEQ ID NO: 52 | | | pET32a-TRXtag |
| F_TRX-BT4720_His | GTACCGACGACGACGACAAGAT | B. thetaiotaomicron VPI- | For cloning into |
| SEQ ID NO: 53 | GGAAGAATTTGAGTTGTC | 5482 genomic DNA | pET32a-TRXtag |
| R_TRX-BT4720-His | CAATGATGATGATGATGATGTC | B. thetaiotaomicron VPI- | For cloning into |
| SEQ ID NO: 54 | CGTTATTCATTATCCATTC | 5482 genomic DNA | pET32a-TRXtag |

### Genetic Manipulation of Bt.

Deletion mutants were constructed using the pSIE1 vector. Briefly, ˜750 base pair regions flanking genes of interest were cloned into pSIE1. Vectors were conjugated into Bt, and positive conjugants were identified by selection on BHI plates containing gentamicin and erythromycin. Counterselection was performed on BHI plates in the presence or absence of 125 ng/ml anhydrotetracycline (aTc). Mutants were identified by PCR prior to whole genome sequencing. Complemented strains were made using vectors.

### OMV Isolation.

OMVs were purified by ultracentrifugation from cell-free culture supernatants. Briefly, 50 mL of Bt culture grown to late stationary phase was centrifuged twice at 6,500 rpm at 4° C. for 10 min. Supernatants were filtered using a 0.22-μm-pore membrane (Millipore) to remove residual cells. The filtrate was subjected to ultracentrifugation at 200,000×g for 2 h (Optima L-100 XP ultracentrifuge; Beckman Coulter). Supernatants were discarded, and pellets were resuspended in PBS standardized to OD600. When performing MS analysis, purified OMV preparations were lyophilized.

### Subcellular Fractionation.

Total Membrane (TM) preparations were isolated by cell lysis and ultracentrifugation. Briefly, late stationary phase cultures were harvested by centrifugation at 6,500 rpm at 4° C. for 10 min. The pellets were gently resuspended in a mixture of phosphate-buffered saline (PBS) containing complete EDTA-free protease inhibitor mixture (Roche Applied Science). Cells were then lysed using two passes through a cell disruptor at 35 kPa. Next, centrifugation at 8,500 rpm at 4° C. for 8 min was performed to remove unbroken cells. Total membranes were collected by ultracentrifugation at 200,000×g for 1 h at 4° C. Supernatants were discarded, and pellets were resuspended in PBS standardized to OD600. TM fractions were lyophilized for MS analysis.

### SDS-PAGE and Western Blot Analyses.

Total membrane and vesicle fractions were analyzed by standard 10% Tris-glycine SDS-PAGE. Samples were normalized by OD600, and equivalent volumes were loaded onto an SDS-PAGE gel. Coomassie Blue staining was employed to analyze protein profiles. When applicable, samples were transferred onto a nitrocellulose membrane for western blot analysis. Membranes were blocked using Tris-buffered saline (TBS)-based Odyssey blocking solution (LI-COR). Primary antibodies utilized herein were rabbit polyclonal anti-His (ThermoFisher) and mouse monoclonal anti-FLAG (Sigma). Secondary antibodies used were IRDye anti-rabbit 780 (LI-COR). Imaging was performed using an Odyssey CLx scanner (LI-COR).

### Lipopolysaccharide (LPS) Silver Stain.

Abundance of LPS was measured. Briefly, samples were normalized by OD600, then diluted and treated with Proteinase K, prior to loading equal amounts onto a 15% SDS-PAGE gel. After running, gels were fixed overnight in 200 mL of 40% ethanol in 5% acetic acid. Next, gels were oxidized for 5 min in 100 mL of 0.7% fresh periodic acid in 40% ethanol and 5% acetic acid. Upon completion, gels underwent three washes (15 min each) in milliQ H2O. The gels were then stained for 10 min in the dark with 28 mL 0.1 M NaOH, 2 mL NH₄OH, 5 mL 20% AgNO₃, and 115 milliQ H₂O. Gels underwent three additional washes prior to developing in 200 mL H2O with 10 mg citric acid and 100 μL formaldehyde.

### OMV Reporter Screen.

Transposon mutagenesis was performed by adapting a protocol on Bt constitutively expressing BACOVA_04502 (Inulinase; INL) fused to Nanoluciferase (NLuc) at the C terminus. Briefly, INL-NLuc was cloned in the pWW3867 vector and expressed in Bt. Next, cells were conjugated with pSAM-Bt_Tet (Table 5) to create the transposon mutant library. Selection was performed on BHI agar plates containing 25 μg/mL erythromycin and 10 μg/mL tetracycline. Upon plating, individual colonies were isolated and transferred to 200 UL of BHI media in clear, round-bottom 96-well plates (Corning) and incubated for 24 h. After incubation, samples were subcultured and diluted (1:20) in the same volume of BHI media and transferred to a second 96-well plate for a 20-h incubation. Next, the OD600 of cultures was measured in each plate with a BioTek microplate reader. Plates then underwent centrifugation at 4,000 rpm to pellet cells. Supernatants were collected and transferred to 0.22 μm hydrophilic low protein binding 96-well filter plates (Millipore) and centrifuged at 4,000 rpm to remove residual cells. Quantification of OMV production in 96-well plate format was done by performing Nano-Glo assays using the Nano-Glo Live Cell Assay System (Promega). Briefly, 100 μL of filtered supernatants was transferred to white bottom 96-well plates (Corning) and 20 μL of Nano-Glo Live Cell Reagent was added to each well. Plates were shaken for 20 s in a BioTek microplate reader prior to quantifying luminescent output. Luminescence was normalized to OD600. Transposon mutants displaying a >1.5-fold increase relative to the wild type were considered hypervesiculating strains, while those displaying <0.5-fold decrease were deemed hypovesiculating strains. These candidates of interest then underwent secondary screening to further characterize the phenotype. Transposon insertions from candidates of interest were identified by genomic DNA extraction and sequencing.

### Protein Expression for In Vitro Pulldown Assay.

Here, in vitro pulldown assays were adapted. Briefly, the N-terminal 40 amino acids of Dma1 were cloned into the pGSTag vector to serve as a bait protein, while Das1 was cloned into the pET32a-TRXtag vector to function as the prey protein. Constructs were expressed in E. coli BL21 by induction of mid-log phase cells for 3 h with 1 mM isopropyl-β-D-thio-galactopyranoside (IPTG). Cells were extracted in 20 mM Tris-HCl pH 8.0, 300 mM NaCl, and supplemented with protease inhibitor. Cells were lysed by a single passage through a cell disruptor at 35 kPa. After disruption, lysates were incubated in 1% n-dodecyl-β-D-maltoside (DDM) for at least 3 h. Protein stability was checked by affinity purifying GST fusion proteins with glutathione-Sepharose 4B resin and TRX-His fusion proteins with Ni-nitrilotriacetic acid (NTA) resin, followed by SDS-PAGE. Upon confirmation of stability, lysates from the bait and prey proteins were used during the pulldown assays.

### In Vitro Pulldown Assay.

Pulldown assays were performed by collecting lysates from cells expressing either bait or prey proteins. Next, lysates containing the GST-fused bait protein were mixed in a 1:1 ratio with those of the TRX-His tagged prey proteins and incubated in the presence of 10% glycerol, protein extraction buffer, and glutathione Sepharose resin at 4° C. overnight while shaking. Controls were included to rule out nonspecific interactions with the affinity tags. Next, an aliquot of the mixture was collected prior to passage through a column (Input). The resin underwent three successive washes with extraction buffer supplemented with 0.2% DDM. Bound proteins were eluted in 50 mM Tris-HCl pH 8.0 and 10 mM reduced glutathione (Output). Equivalent amounts of sample from the input and output were analyzed by western blotting with mouse monoclonal GST (Millipore Sigma) and rabbit polyclonal His (Invitrogen) antibodies.

### Phylogenetic Analysis of the Dma Family.

Whole-genome fasta files were obtained from NCBI Assembly on Jun. 28, 2023. Genomes were annotated for open reading frames with prokka v1.14.6 using the command “prokka $ {ID}_*. fna--outdir $ {ID}--locustag $ {ID}--mincontiglen 500--prefix $ {ID}--force--notrna--norrna” {24642063}. The core genome alignment of the 29 genomes was created using panaroo v1.2.10 on the .gff files from prokka with the command “panaroo-i $ {indir}/*.gff-o $ {outdir}--clean-mode moderate-a core-c.8-f.5--core_threshold.99-t 12--search_radius 10--refind_prop_match 100-t $ {SLURM_CPUS_PER_TASK}” {32698896}. Alignment of the 29 core genes identified by panaroo was performed using mafft v7 {23329690}. The core genome alignment from panaroo was constructed into a maximum likelihood phylogenetic tree using RAxML v8.2.12 with the command “raxmlHPC-s core_gene_alignment.aln-n EP_raxml-m GTRGAMMA-f a-T 4-N 100-p 12345-x 54321” {24451623}. Identification of Dma1, Dma2, and Dma3 homologs outside of B. thetaiotamicron was performed with the NCBI tblastn webserver using the protein sequences for each gene as query and the whole-genome fasta files as the subject with 95% query cover threshold as a positive result for gene presence. AlphaFold structural predictions were used to confirm genes identified during the blast search. The newick file was viewed and analyzed with metadata on Dma1, Dma2, Dma3, related gene information in iTOL webserver {33885785}.

### Data, Materials, and Software Availability.

Mass Spectrometry Proteomics data has been deposited in the Proteome Xchange Consortium via the PRIDE partner repository with the dataset identifier PXD043360. RNA sequencing data has been deposited with links to BioProject accession number PRJNA994135 in the NCBI BioProject database.

### Extended Methods

### RNA Sequencing Sample Collection, Library Preparation, and Analysis.

WT and Δdma1 were grown overnight in BHI media before being diluted to the equivalent of OD 0.1 in 10 mL and grown anaerobically for 4 h at 37° C. Four individual overnight and 10-mL culture biological replicates were prepared. Cultures were normalized, and an amount of culture equivalent to an OD600 of 4.0 was pelleted for 90s at 8,000 rpm. Pellets were resuspended on ice in 1 mL TRIzol (Invitrogen) with 10 μL of 5 mg/mL glycogen. Samples were flash frozen and stored at −80° C. until extraction. Prior to extraction, samples were thawed on ice, then pelleted, and supernatants were treated with chloroform. RNA was extracted from the aqueous phase using the RNeasy minikit (Qiagen, Inc.), and RNA quality was checked by agarose gel electrophoresis and Δ260/A280 measurements. RNA was stored at −80° C. with SUPERase-IN RNase inhibitor (Life Technologies) until library preparation.

RNA sequencing prep (RNA-Seq) was performed. Briefly, 400 ng of total RNA from each sample was used for generating cDNA libraries following RNAtag-Seq protocol. PCR amplified cDNA libraries were sequenced on an Illumina NextSeq500, obtaining a high-sequencing depth (over 7 million reads per sample). RNA-Seq data was analyzed using in-house-developed analysis pipeline Aerobio. Raw reads are demultiplexed by 5′ and 3′ indices, trimmed to 59 base pairs, and quality filtered (96% sequence quality>Q14). Filtered reads are mapped to the corresponding reference genomes using bowtie2 with the -very-sensitive option (-D 20-R 3-N 0-L 20-i S, 1, 0.50). Mapped reads are aggregated by feature Count and differential expression is calculated with DESeq2. In each pair-wise differential expression comparison, significant differential expression is filtered based on two criteria: |log 2foldchange|>1 and adjusted p-value (padj) 0.95). BioProject ID PRJNA994135.

### Sample Preparation for Proteomic Analysis.

WT, Δdas1, Δdma1, and Δdas1-dma1 were grown overnight anaerobically in 2 mL of BHI media prior to being diluted into 50 mL and grown for 20 h. Whole cells, total membranes, and vesicles were collected from each strain. Four individual biological replicates of each fraction were performed for each strain. Samples were lyophilized in preparation for MS analysis.

Lyophilized protein samples were solubilized in 4% SDS, 100 mM Tris pH 8.5 by boiling them for 10 min at 95° C. The protein concentrations were assessed using a bicinchoninic acid protein assay (Thermo Fisher Scientific) and 100 μg of each biological replicate prepared for digestion using Micro S-traps (Protifi, USA) according to the manufacturer's instructions. Briefly, samples were reduced with 10 mM DTT for 10 mins at 95° C. and then alkylated with 40 mM IAA in the dark for 1 hour. Samples were acidified to 1.2% phosphoric acid and diluted with seven volumes of S-trap wash buffer (90% methanol, 100 mM Tetraethylammonium bromide pH 7.1) before being loaded onto S-traps and washed 3 times with S-trap wash buffer. Samples were then incubated with Trypsin (1:100 protease: protein ratio, in 100 mM Tetraethylammonium bromide pH 8.5) overnight at 37° C. before being collected by centrifugation with washes of 100 mM Tetraethylammonium bromide pH 8.5, followed by 0.2% formic acid followed by 0.2% formic acid/50% acetonitrile. Samples were then dried down and further cleaned up using homemade C18 Stage 1,2 tips to ensure the removal of any particulate matter.

### Reverse Phase Liquid Chromatography-Mass Spectrometry.

C₁₈ purified digests were re-suspended in Buffer A* (2% acetonitrile, 0.01% trifluoroacetic acid) and separated on a Ultimate 3000 UPLC (Thermo Fisher Scientific) with a two-column chromatography set up composed of a PepMap100 C18 20 mm×75 μm trap and a PepMap C18 500 mm×75 μm analytical column (Thermo Fisher Scientific). Digests were loaded on to the trap column at 5 μL/min for 6 minutes with Buffer A (0.1% formic acid, 2% DMSO) then infused into a Orbitrap 480 TM at 300 nl/minute via the analytical column. 95-minute runs were undertaken by altering the buffer composition from 2% Buffer B to 28% B over 70 minutes, then from 25% B to 40% B over 4 minutes, then from 40% B to 80% B over 3 minutes. The composition was held at 80% B for 2 minutes, and then dropped to 2% B over 1 minutes before being held at 2% B for another 10 minutes. The Orbitrap 480 TM Mass Spectrometer was operated in a data-dependent mode automatically switching between the acquisition of a single Orbitrap MS scan (300-1600 m/z, maximal injection time of 25 ms, an AGC set to a maximum of 300% and a resolution of 120 k) every 3 seconds and Orbitrap MS/MS HCD scans of precursors (using a stepped NCE of 28,32,40%, maximal injection time of 80 ms, an AGC set to a maximum of 300% and a resolution of 30 k). Identification and LFQ analysis were accomplished using MaxQuant (v1.6.17.0)₃. Data was searched against the B. thetaiotaomicron reference proteome (Uniprot: UP000001414) allowing for oxidation on Methionine. The LFQ and “Match Between Run” options were enabled to allow comparison between samples. Maxquant search results were processed using Perseus (version 1.6.0.7)₃ with missing values imputed based on the total observed protein intensities with a range of 0.3 σ and a downshift of 1.8 σ. Statistical analysis was undertaken in Perseus using two-tailed unpaired T-tests between groups.

### MS Data Analysis.

Identification and LFQ analysis were accomplished using Max-Quant (v2.0.2.0)₈ using Bacteroides thetaiotaomicron VPI-5482 proteome (Uniprot: UP000001414) allowing for oxidation on Methionine. Prior to MaxQuant analysis dataset acquired on the Fusion Lumos were separated into individual FAIMS fractions using the FAIMS MzXML Generator9. The LFQ and “Match Between Run” options were enabled to allow comparison between samples. The resulting data files were processed using Perseus (v1.4.0.6)₁₀ to filter proteins not observed in at least four biological replicates of a single group. ANOVA and Pearson correlation analyses were performed to compare groups. Predicted localization and topology analysis for proteins identified by MS were performed using UniProt11, PSORT12, SignalP13 and PULDB14.

LC-MS Analysis of Lipids from TM and OMVs.

WT, Δdma1, and its complemented strain were grown overnight anaerobically in 5 mL of BHI media prior to being diluted into 140 mL and grown for 20 h. TMs and OMVs were collected from each strain. Four individual biological replicates of each fraction were performed for each strain. Total lipids were extracted according to Bligh and Dyer chloroform:methanol method. Briefly, 2 volumes of methanol, 1 volume of chloroform, and 0.8 volumes of Milli-Q water were added to 1 volume of PBS-resuspended sample in solvent-resistant glass tubes. Contents were mixed for 2 min by vortexing, and 1 volume of chloroform was added to the mixture. Samples were vortexed for another minute, then centrifugated for 5 min at 4,000 rpm. After centrifugation, the bottom phase (organic) was recovered using a glass Pasteur pipette and stored in solvent-sealed vials at −80° C. until lipid analysis by LC-MS.

Untargeted LC/MS analyses were conducted on an Agilent 6550 A QTOF instrument with an Agilent 1290 high-performance liquid chromatograph (HPLC) with an autosampler, operated using Agilent MassHunter software (Santa Clara, CA, USA). Separation of the total lipid extracts was achieved using a Thermo Fisher (Waltham, MA, USA) BETASIL C18 column (100×2.1 mm, 5 μm) at a flow rate of 300 μL/min at room temperature. The mobile phase contained 5 mM ammonium formate (pH 5.0) both in solvent A, acetonitrile: water (60:40, vol/vol), and solvent B, isopropanol: acetonitrile (90:10, vol/vol). A gradient elution was applied in the following manner: 68% A, 0 to 1.5 min; 68 to 55% A, 1.5 to 4 min; 55 to 48% A, 4 to 5 min; 48 to 42% A, 5 to 8 min; 42 to 34% A, 8 to 11 min; 34 to 30% A, 11 to 14 min; 30 to 25% A, 14 to 18 min; 25 to 3% A, 18 to 23 min; 3 to 0% A, 25 to 30 min; 0% A, to 35 min; 68% A, 35 to 40 min. Both the positive-ion and negative-ion electrospray ionization (ESI) MS scans were acquired in the mass range of 200 to 2,000 Da at a rate of 2 scans/min. High-resolution (R=100,000 at m/z 400) mass spectrometric analyses of the lipid extracts were also conducted on a Thermo LTQ Orbitrap Velos. Lipids were loop injected into the ESI ion source using a built-in syringe pump which was set to continuously deliver a flow of 20 μL/min methanol with 0.5% NH₄OH. The scanned mass spectra were recalibrated internally with a known mass, namely, 13:0/15:0 PE at m/z 634.4453. Linear ion trap (LIT) multistage MS (MS″) spectra were obtained for structural identification as described previously.

### Negative Staining and Analysis by Transmission Electron Microscopy.

For quantitative analyses at the ultrastructural level, 200 mesh formvar/carbon-coated copper grids (Ted Pella Inc., Redding CA) were coated with 50 μg/ml poly-L-lysine (Sigma, St Louis, MO) for 10 min at 37° C. Excess fluid was removed, and grids were allowed to air dry. Poly-L-lysine coating allowed for even distribution of material across the grid. Bacterial OMVs were fixed with 1% glutaraldehyde (Ted Pella Inc.) and allowed to absorb onto freshly glow discharged poly-L-lysine-coated grids for 10 min. Grids were then washed in dH²O and stained with 1% aqueous uranyl acetate (Ted Pella Inc.) for 1 min. Excess liquid was gently wicked off and grids were allowed to air dry. Samples were viewed on a JEOL 1200EX transmission electron microscope (JEOL USA, Peabody, MA) equipped with an AMT 8-megapixel digital camera (Advanced Microscopy Techniques, Woburn, MA). Each OMV sample was processed in triplicate (3 grids). Ten random images were taken at a magnification of 25,000× from various areas of the grid with a total of 90 images for each sample, and the number of OMV in each image was quantified.

What is claimed is:

1. An engineered Bacteroides thetaiotaomicron (Bt) strain, comprising at least one mutated dual membrane-spanning anti-sigma factor (Dma) protein.

2. The engineered Bt strain of claim 1, wherein the at least one mutated Dma protein is selected from Dma1, Dma2, and Dma3.

3. The engineered Bt strain of claim 1, wherein the at least one mutated Dma protein is selected from a BT_4721 mutation and a BT_1558 mutation.

4. The engineered Bt strain of claim 1, wherein the at least one mutated Dma protein comprises a deletion mutant.

5. The engineered Bt strain of claim 4, wherein the deletion mutant is Δdma1.

6. The engineered Bt strain of claim 1, further comprising a Dma-associated sigma factor (Das) protein.

7. The engineered Bt strain of claim 6, wherein the Das protein is selected from Das1, Das2, and Das3.

8. A method for overproducing outer membrane vesicles (OMVs), the method comprising:
modifying a Bacteroides thetaiotaomicron (Bt) strain to produce an engineered Bt strain, wherein the modification comprises at least one mutated dual membrane-spanning anti-sigma factor (Dma) protein.

9. The method of claim 8, wherein the at least one mutated Dma protein is selected from Dma1, Dma2, and Dma3.

10. The method of claim 8, wherein the at least one mutated Dma protein is selected from a BT_4721 mutation and a BT_1558 mutation.

11. The method of claim 8, wherein the at least one mutated Dma protein comprises a deletion mutant.

12. The method of claim 11, wherein the deletion mutant is Δdma1.

13. The method of claim 8, further comprising a Dma-associated sigma factor (Das) protein.

14. The method of claim 13, wherein the Das protein is selected from Das1, Das2, and Das3.

15. A method of delivering a therapeutic compound to a gut microbiome of a subject, the method comprising:
culturing an engineered Bacteroides thetaiotaomicron (Bt) strain in a growth medium to overproduce outer membrane vesicles (OMVs);
isolating the OMVs from the growth medium;
loading a target therapeutic compound into the OMVs; and
administering the loaded OMVs to the subject.

16. The method of claim 15, wherein the subject has at least one of lactose intolerance, phenylketonuria, inflammatory bowel disease, and a chronic intestinal condition.

17. The method of claim 15, wherein the engineered Bt strain comprises at least one of:
a mutated dual membrane-spanning anti-sigma factor (Dma) protein selected from Dma1, Dma2, and Dma3;
a Dma protein mutation selected from a BT_4721 mutation and a BT_1558 mutation; and
a Dma protein deletion mutant.

18. The method of claim 17, wherein the Dma protein deletion mutant is Δdma1.

19. The method of claim 17, further comprising a Dma-associated sigma factor (Das) protein.

20. The method of claim 19, wherein the Das protein is selected from Das1, Das2, and Das3.


SEQUENCE LISTING (70 sequences)

SEQ ID NO: 1 - DNA, 21 bp, synthetic construct
  source 1..21: mol_type = other DNA
gtcttcacactcgaagatttc

SEQ ID NO: 2 - DNA, 59 bp, synthetic construct
  source 1..59: mol_type = other DNA
tcgagctaatcagctaggatttagtgatgatgatgatgatgacccgccagaatgcgttc

SEQ ID NO: 3 - DNA, 49 bp, synthetic construct
  source 1..49: mol_type = other DNA
tccaaatctgtttttaaagaatgaagataaataaattcttaataagcgg

SEQ ID NO: 4 - DNA, 56 bp, synthetic construct
  source 1..56: mol_type = other DNA
aaatcttcgagtgtgaagacagatcctcctcctcctttcttagcgcttagataatg

SEQ ID NO: 5 - DNA, 21 bp, synthetic construct
  source 1..21: mol_type = other DNA
gtcttcacactcgaagatttc

SEQ ID NO: 6 - DNA, 56 bp, synthetic construct
  source 1..56: mol_type = other DNA
tcgagctaatcagctaggatttagtgatgatgatgatgatgacccgccagaatgcg

SEQ ID NO: 7 - DNA, 36 bp, synthetic construct
  source 1..36: mol_type = other DNA
gcggccgctctagaactagtcgccttcgtcgttatg

SEQ ID NO: 8 - DNA, 41 bp, synthetic construct
  source 1..41: mol_type = other DNA
tttctcttccatatctctacttgcttatacacgtgtttacc

SEQ ID NO: 9 - DNA, 48 bp, synthetic construct
  source 1..48: mol_type = other DNA
gtaaacacgtgtataagcaagtagagatatggaagagaaagaattatg

SEQ ID NO: 10 - DNA, 40 bp, synthetic construct
  source 1..40: mol_type = other DNA
gattagcattatgaggatccattgctgatcattgggtatg

SEQ ID NO: 11 - DNA, 50 bp, synthetic construct
  source 1..50: mol_type = other DNA
gcggccgctctagaactagttggtcaggatctcttttatttctcttacct

SEQ ID NO: 12 - DNA, 32 bp, synthetic construct
  source 1..32: mol_type = other DNA
atctctacctatccgttattcattatccattc

SEQ ID NO: 13 - DNA, 53 bp, synthetic construct
  source 1..53: mol_type = other DNA
aataacggataggtagagattagaacccggtgttgcttatttctttgatgatg

SEQ ID NO: 14 - DNA, 44 bp, synthetic construct
  source 1..44: mol_type = other DNA
aagattagcattatgaggatccggtgacgtatgtttcagtgttc

SEQ ID NO: 15 - DNA, 41 bp, synthetic construct
  source 1..41: mol_type = other DNA
atatagctcaactatattgattgcttatacacgtgtttacc

SEQ ID NO: 16 - DNA, 51 bp, synthetic construct
  source 1..51: mol_type = other DNA
gtaaacacgtgtataagcaatcaatatagttgagctatatattttaaaagc

SEQ ID NO: 17 - DNA, 37 bp, synthetic construct
  source 1..37: mol_type = other DNA
gcggccgctctagaactagtcaggcacagtgtcatag

SEQ ID NO: 18 - DNA, 49 bp, synthetic construct
  source 1..49: mol_type = other DNA
cgctgatgccgggatgactgtccttataaaaatgaaaaaaacacttatg

SEQ ID NO: 19 - DNA, 33 bp, synthetic construct
  source 1..33: mol_type = other DNA
ttttttcatttttataaggacagtcatcccggc

SEQ ID NO: 20 - DNA, 33 bp, synthetic construct
  source 1..33: mol_type = other DNA
gattagcattatgaggatcccgtgctgcagctg

SEQ ID NO: 21 - DNA, 35 bp, synthetic construct
  source 1..35: mol_type = other DNA
gcggccgctctagaactagtgctttcagcgggatg

SEQ ID NO: 22 - DNA, 41 bp, synthetic construct
  source 1..41: mol_type = other DNA
acaccctctatgccaaacaatgaaataaccgatctgtttcg

SEQ ID NO: 23 - DNA, 37 bp, synthetic construct
  source 1..37: mol_type = other DNA
gaaacagatcggttatttcattgtttggcatagaggg

SEQ ID NO: 24 - DNA, 35 bp, synthetic construct
  source 1..35: mol_type = other DNA
gattagcattatgaggatccttggatacccgcaag

SEQ ID NO: 25 - DNA, 36 bp, synthetic construct
  source 1..36: mol_type = other DNA
gcggccgctctagaactagtccggtcatcctttacg

SEQ ID NO: 26 - DNA, 49 bp, synthetic construct
  source 1..49: mol_type = other DNA
taaataattaaacagtcacctttagttaaagttttcatacttgttaaac

SEQ ID NO: 27 - DNA, 43 bp, synthetic construct
  source 1..43: mol_type = other DNA
gtatgaaaactttaactaaaggtgactgtttaattatttacag

SEQ ID NO: 28 - DNA, 33 bp, synthetic construct
  source 1..33: mol_type = other DNA
gattagcattatgaggatccagaggcgaatgcg

SEQ ID NO: 29 - DNA, 38 bp, synthetic construct
  source 1..38: mol_type = other DNA
gcggccgctctagaactagttaggaaatcctacggtag

SEQ ID NO: 30 - DNA, 43 bp, synthetic construct
  source 1..43: mol_type = other DNA
agttatttataaaagttttgatttgttcctctctttttaattc

SEQ ID NO: 31 - DNA, 50 bp, synthetic construct
  source 1..50: mol_type = other DNA
ttaaaaagagaggaacaaatcaaaacttttataaataactaaacctaaac

SEQ ID NO: 32 - DNA, 33 bp, synthetic construct
  source 1..33: mol_type = other DNA
gattagcattatgaggatccacctgttcctcgc

SEQ ID NO: 33 - DNA, 37 bp, synthetic construct
  source 1..37: mol_type = other DNA
gcggccgctctagaactagtgaagtgctgtttttgcc

SEQ ID NO: 34 - DNA, 43 bp, synthetic construct
  source 1..43: mol_type = other DNA
aaaaaacatttttatagtatatctagtaattatagaaacactc

SEQ ID NO: 35 - DNA, 56 bp, synthetic construct
  source 1..56: mol_type = other DNA
tgtttctataattactagatatactataaaaatgttttttaatgagtactatttac

SEQ ID NO: 36 - DNA, 42 bp, synthetic construct
  source 1..42: mol_type = other DNA
gattagcattatgaggatcctctttctcttccatatctctac

SEQ ID NO: 37 - DNA, 41 bp, synthetic construct
  source 1..41: mol_type = other DNA
tccaaatctgtttttaaagaatggaagaatttgagttgtcg

SEQ ID NO: 38 - DNA, 71 bp, synthetic construct
  source 1..71: mol_type = other DNA
tcgagctaatcagctaggatctagtgatgatgatgatgatgacctccgttattcattatccattcttttac

SEQ ID NO: 39 - DNA, 40 bp, synthetic construct
  source 1..40: mol_type = other DNA
ccaaatctgtttttaaagaatggaagagaaagaattatgg

SEQ ID NO: 40 - DNA, 74 bp, synthetic construct
  source 1..74: mol_type = other DNA
tcgagctaatcagctaggatctaatgatgatgatggtgatgatgatgatgatgaccataagtaagtcggatacc

SEQ ID NO: 41 - DNA, 71 bp, synthetic construct
  source 1..71: mol_type = other DNA
tttctcttccatatctctacctagtgatgatgatgatgatgacctccgttattcattatccattcttttac

SEQ ID NO: 42 - DNA, 41 bp, synthetic construct
  source 1..41: mol_type = other DNA
catcatcactaggtagagatatggaagagaaagaattatgg

SEQ ID NO: 43 - DNA, 19 bp, synthetic construct
  source 1..19: mol_type = other DNA
atggattacaaggacgatg

SEQ ID NO: 44 - DNA, 39 bp, synthetic construct
  source 1..39: mol_type = other DNA
atccataattctttctcttctccagatttatcatcatcg

SEQ ID NO: 45 - DNA, 41 bp, synthetic construct
  source 1..41: mol_type = other DNA
acgatgatgataaatctggagaagagaaagaattatggatg

SEQ ID NO: 46 - DNA, 74 bp, synthetic construct; identical to SEQ ID NO: 40
  source 1..74: mol_type = other DNA

SEQ ID NO: 47 - DNA, 15 bp, synthetic construct
  source 1..15: mol_type = other DNA
gcagaacgtcgcgaa

SEQ ID NO: 48 - DNA, 15 bp, synthetic construct
  source 1..15: mol_type = other DNA
agatccacgcggaac

SEQ ID NO: 49 - DNA, 41 bp, synthetic construct
  source 1..41: mol_type = other DNA
atctggttccgcgtggatctatggaagagaaagaattatgg

SEQ ID NO: 50 - DNA, 35 bp, synthetic construct
  source 1..35: mol_type = other DNA
agaatttcgcgacgttctgcctacctctccacagg

SEQ ID NO: 51 - DNA, 22 bp, synthetic construct
  source 1..22: mol_type = other DNA
catcatcatcatcatcattgag

SEQ ID NO: 52 - DNA, 15 bp, synthetic construct
  source 1..15: mol_type = other DNA
cttgtcgtcgtcgtc

SEQ ID NO: 53 - DNA, 40 bp, synthetic construct
  source 1..40: mol_type = other DNA
gtaccgacgacgacgacaagatggaagaatttgagttgtc

SEQ ID NO: 54 - DNA, 41 bp, synthetic construct
  source 1..41: mol_type = other DNA
caatgatgatgatgatgatgtccgttattcattatccattc

SEQ ID NO: 55 - protein, 12 aa, synthetic construct
EKEEVEPVEETK

SEQ ID NO: 56 - protein, 9 aa, synthetic construct
DKLHIPAEK

SEQ ID NO: 57 - protein, 7 aa, synthetic construct
LHIPAEK

SEQ ID NO: 58 - protein, 21 aa, synthetic construct
QANQVVDMEHHQPISFGLSVR

SEQ ID NO: 59 - protein, 18 aa, synthetic construct
GFSVETGLTYTLLSSDAK

SEQ ID NO: 60 - protein, 9 aa, synthetic construct
LHYLGIPLK

SEQ ID NO: 61 - protein, 8 aa, synthetic construct
ANWNFLDK

SEQ ID NO: 62 - protein, 9 aa, synthetic construct
ANWNFLDKK

SEQ ID NO: 63 - protein, 22 aa, synthetic construct
ETVKPLQFSVSGAVGAQFNATK

SEQ ID NO: 64 - protein, 24 aa, synthetic construct
RVGIYVEPGVAYFFDDGSDVQTIR

SEQ ID NO: 65 - protein, 23 aa, synthetic construct
VGIYVEPGVAYFFDDGSDVQTIR

SEQ ID NO: 66 - protein, 14 aa, synthetic construct
KENPFNFNIQAGIR

SEQ ID NO: 67 - protein, 13 aa, synthetic construct
ENPFNFNIQAGIR

SEQ ID NO: 68 - protein, 425 aa, synthetic construct
XMEEKELWMNKLKEKLGDYSEPLPASGWEQLEKELMPPVERKIYPYRKWTVAAAAVILLALGSSVSLYFLGTPAADEIRHAKTPALASVPDVLPDAQQPDMTGTTIEPVVRPVVKNRIAKAERNIPQPTANIDEPVKKEEQPSELNAQTGDRKEKEEVEPVEETKAIRHKPADTEQPXXRNKPRRPSSRDKLHIPAEKASSQKGXTWSMGLSVGNSGGASTELGSGIPSXXYMSRVSMVSVSNGLLSIPNDQQLVFEDGVPYLRQXANQVVDMEHHQPISFGLSVRKSLAKGFSVETGLTYTLLSSDAKFADSDQKTEQKLHYLGIPLKANWNFLDKKLFTLYVSGGGMIEKCVYGKLGTEKETVKPLQFSVSGAVGAQFNATKRVGIYVEPGVAYFFDDGSDVQTIRKENPFNFNIQAGIRLTY

SEQ ID NO: 69 - protein, 425 aa, synthetic construct
MMKEDEKWIKAFKDKLEDYSEPMPASGWERLERELMPVTEKRIYPYRRWAVAAAAVVLVXLTTAVSLYFLNSPVADEIRYATAPSLAVNPDVLPEPALPDVQVAVSEPVXKPVGTTSINPVSGYLAKNTDPVIVPEVSSLVEKRPEAVTEEKRSEPQQEAIAXXXAIEKKESATAQPPKRKEARRPSGKDKYQLPIGDSSAKRGGKWSMGVGIGNGGGLPTNGSENFAPRPMTNRVDLMTIMNGAVSIPADQEVIFEEGVPYLKSNTTAVVDYEHHQPVSFGLSVRKSLPKGFSVETGLTYTLLSSDIKRQGDTKMQSQKLHYIGIPVRGNWNFLEKKYFTLYVSAGGMVEKCVYGKLADDKVNVKPLQFSVAGAVGAQFNATDHVGLYVEPGVSYFFDDGSKVQTIRKERPCNFNLQAGLRFTY

SEQ ID NO: 70 - protein, 425 aa, synthetic construct
XMEEDELWINALKXKLEDYSEPXPASGWERLERELMPPTERRIYPYRRWAVAAAAVXLLXLGSAVSLYFLNSPAADEIRHAKAPALASNPDVLPXAAQPDMQGATIEPVXRPVGKNRIAKAERNIAQNTANXDEPEKKEEQEKRLXAQTEXRREEEXQEAXAXXXAIRHKEAATAQPXXRNEARRPSGRDKLQIPAEDASAQRGXKWSMGLGXGNGGGAPTXGGENIAPXXMMNRVDXMSXMNGALSIPADQXLXFEXGVPYLRQXANAVVDMEHHQPXSFGLSVRKSLAKGFSVETGLTYTLLSSDAKRADDDQKQEQKLHYIGIPLRANWNFLXKKLFTLYVSAGGMXEKCVYGKLADXKENVKPLQFSVAGAVGAQFNATDRVGIYVEPGVAYFFDDGSDVQTIRKERPCNFNIQAGIRLTY